SEQUENCE LISTING (160 sequences)

SEQ ID NO: 1 - protein, 124 aa, Artificial Sequence
  Description of Artificial Sequence Synthetic polypeptide
AVQLQQSGPVRVKPGASVKMSCKASGYPFTDYYMNWVKQSHGRSLDWIGVFNPYNGDTRYTQKFKDKATLTIDKSSSTAYMELNSLTSEDSAVYYCARLRLLYDTSEYYVMDYWGQGTSVTVSS

SEQ ID NO: 2 - protein, 9 aa, Artificial Sequence
  Description of Artificial Sequence Synthetic peptide
YPFTDYYMN

SEQ ID NO: 3 - protein, 15 aa, Artificial Sequence
  Description of Artificial Sequence Synthetic peptide
WIGVFNPYNGDTRYT

SEQ ID NO: 4 - protein, 16 aa, Artificial Sequence
  Description of Artificial Sequence Synthetic peptide
RLRLLYDTSEYYVMDY

SEQ ID NO: 5 - protein, 106 aa, Artificial Sequence
  Description of Artificial Sequence Synthetic polypeptide
ENVLTQSPAIMSASPGEKVTMTCSASSSVNYMHWYQQKSNTSPKLWIYDTSKLASGVPGRFSGSGSGNSYSLTISSMEAEDVATYFCFQGSGYPYTFGGGTKLEIK

SEQ ID NO: 6 - protein, 7 aa, Artificial Sequence
  Description of Artificial Sequence Synthetic peptide
SSVNYMH

SEQ ID NO: 7 - protein, 11 aa, Artificial Sequence
  Description of Artificial Sequence Synthetic peptide
LWIYDTSKLAS

SEQ ID NO: 8 - protein, 8 aa, Artificial Sequence
  Description of Artificial Sequence Synthetic peptide
FQGSGYPY

SEQ ID NO: 9 - protein, 116 aa, Artificial Sequence
  Description of Artificial Sequence Synthetic polypeptide
QVQLQQSGAELMKPGASVKLACKATGYTFTAYWIEWVKQRPGHGLEWIGEILPGRGSTNSNEKFKGKATFTADTSSNTVYMQLSSLTTEDSAIFYCARGYRDFDYWGQGTTLTVSS

SEQ ID NO: 10 - protein, 9 aa, Artificial Sequence
  Description of Artificial Sequence Synthetic peptide
YTFTAYWIE

SEQ ID NO: 11 - protein, 14 aa, Artificial Sequence
  Description of Artificial Sequence Synthetic peptide
WIGEILPGRGSTNS

SEQ ID NO: 12 - protein, 8 aa, Artificial Sequence
  Description of Artificial Sequence Synthetic peptide
RGYRDFDY

SEQ ID NO: 13 - protein, 107 aa, Artificial Sequence
  Description of Artificial Sequence Synthetic polypeptide
DIVMTQSQKFMSTTVGDRVSIICKASQNVGTAVAWYQQKPGQSPKVLIYSASNRYTGVPDRFTGSGSGTDFTLTISNMQSEDLADYFCQQYTSYPRTFGGGTKLEIK

SEQ ID NO: 14 - protein, 8 aa, Artificial Sequence
  Description of Artificial Sequence Synthetic peptide
QNVGTAVA

SEQ ID NO: 15 - protein, 11 aa, Artificial Sequence
  Description of Artificial Sequence Synthetic peptide
VLIYSASNRYT

SEQ ID NO: 16 - protein, 8 aa, Artificial Sequence
  Description of Artificial Sequence Synthetic peptide
QQYTSYPR

SEQ ID NO: 17 - protein, 116 aa, Artificial Sequence
  Description of Artificial Sequence Synthetic polypeptide
QVQLQQSGAELMKPGASVKLSCKATGYTFTGYWIEWVKQRPGHGLEWIGEILPGRDSTNSNEKFKGKATFTADTSSNTVYMQLSSLTTEDSAIFYCARGYRDFDYWGQGTTLTVSS

SEQ ID NO: 18 - protein, 9 aa, Artificial Sequence
  Description of Artificial Sequence Synthetic peptide
YTFTGYWIE

SEQ ID NO: 19 - protein, 14 aa, Artificial Sequence
  Description of Artificial Sequence Synthetic peptide
WIGEILPGRDSTNS

SEQ ID NO: 20 - protein, 8 aa, Artificial Sequence
  Description of Artificial Sequence Synthetic peptide
RGYRDFDY

SEQ ID NO: 21 - protein, 107 aa, Artificial Sequence
  Description of Artificial Sequence Synthetic polypeptide
DIVMTQSQKFMSTTVGDRVSIICKASQNVGTAVAWYQQKPGQSPKVLIYSASNRYTGVPDRFTGSGSGTDFTLTISNMQSEDLADYFCQQYSSYPRTFGGGTKLEIK

SEQ ID NO: 22 - protein, 8 aa, Artificial Sequence
  Description of Artificial Sequence Synthetic peptide
QNVGTAVA

SEQ ID NO: 23 - protein, 11 aa, Artificial Sequence
  Description of Artificial Sequence Synthetic peptide
VLIYSASNRYT

SEQ ID NO: 24 - protein, 8 aa, Artificial Sequence
  Description of Artificial Sequence Synthetic peptide
QQYSSYPR

SEQ ID NO: 25 - protein, 128 aa, Artificial Sequence
  Description of Artificial Sequence Synthetic polypeptide
EVQLVESGGGLVQPKGSLQLSCAASGFTFNTYAMHWVRQAPGKGLEWVARIRSKSSNYATYYADSVKDRFTISRDDSQSMLYLQMNNLKTEDTAMYYCVRDGDYYYGSRPSYWYFDVWGTGTTVTVSS

SEQ ID NO: 26 - protein, 9 aa, Artificial Sequence
  Description of Artificial Sequence Synthetic peptide
FTFNTYAMH

SEQ ID NO: 27 - protein, 16 aa, Artificial Sequence
  Description of Artificial Sequence Synthetic peptide
WVARIRSKSSNYATYY

SEQ ID NO: 28 - protein, 18 aa, Artificial Sequence
  Description of Artificial Sequence Synthetic peptide
RDGDYYYGSRPSYWYFDV

SEQ ID NO: 29 - protein, 107 aa, Artificial Sequence
  Description of Artificial Sequence Synthetic polypeptide
DILLTQSPAILSVSPGERVSFSCRASQSIGTNIHWYQQRTNGSPRLLIKYASESISGIPSRFSGSGSGTDFTLSINNVESEDIADYYCQQTNTWPLTFGAGTKLDLK

SEQ ID NO: 30 - protein, 8 aa, Artificial Sequence
  Description of Artificial Sequence Synthetic peptide
QSIGTNIH

SEQ ID NO: 31 - protein, 11 aa, Artificial Sequence
  Description of Artificial Sequence Synthetic peptide
LLIKYASESIS

SEQ ID NO: 32 - protein, 8 aa, Artificial Sequence
  Description of Artificial Sequence Synthetic peptide
QQTNTWPL

SEQ ID NO: 33 - protein, 120 aa, Artificial Sequence
  Description of Artificial Sequence Synthetic polypeptide
EVQLVESGGGLVKPGGSLKLSCAASGFTFSNYVMSWVRQTPEKRLEWVATISDRGSYTYYPDYVKGRFTISRDNAKNNLYLQMSHLKSEDTAMYYCARASYYSNLGFAYWGQGTLVTVSA

SEQ ID NO: 34 - protein, 9 aa, Artificial Sequence
  Description of Artificial Sequence Synthetic peptide
FTFSNYVMS

SEQ ID NO: 35 - protein, 14 aa, Artificial Sequence
  Description of Artificial Sequence Synthetic peptide
WVATISDRGSYTYY

SEQ ID NO: 36 - protein, 12 aa, Artificial Sequence
  Description of Artificial Sequence Synthetic peptide
RASYYSNLGFAY

SEQ ID NO: 37 - protein, 113 aa, Artificial Sequence
  Description of Artificial Sequence Synthetic polypeptide
DIVMSQSPSSLAVSVGEKVTMSCKSSQSLLYSSNQKNYLAWYQQKPGQSPKLLIYWASTRESGVPDRFTGSGSGTDFTLTISSMKAEDLAVYYCQQYYSYPPTFGGGTKLEIK

SEQ ID NO: 38 - protein, 14 aa, Artificial Sequence
  Description of Artificial Sequence Synthetic peptide
QSLLYSSNQKNYLA

SEQ ID NO: 39 - protein, 11 aa, Artificial Sequence
  Description of Artificial Sequence Synthetic peptide
LLIYWASTRES

SEQ ID NO: 40 - protein, 8 aa, Artificial Sequence
  Description of Artificial Sequence Synthetic peptide
QQYYSYPP

SEQ ID NO: 41 - protein, 116 aa, Artificial Sequence
  Description of Artificial Sequence Synthetic polypeptide
QVQLRQSGAELMKPGASVKLSCKATGYTFTGYWIEWVKQRPGHGLEWIGEILPGRGSSNYNEKFKGKATLTADTSSNTVYMQLSSLTIEDSAIYYCARGGRDFDYWGQGTTLTVSS

SEQ ID NO: 42 - protein, 9 aa, Artificial Sequence
  Description of Artificial Sequence Synthetic peptide
YTFTGYWIE

SEQ ID NO: 43 - protein, 14 aa, Artificial Sequence
  Description of Artificial Sequence Synthetic peptide
WIGEILPGRGSSNY

SEQ ID NO: 44 - protein, 8 aa, Artificial Sequence
  Description of Artificial Sequence Synthetic peptide
RGGRDFDY

SEQ ID NO: 45 - protein, 107 aa, Artificial Sequence
  Description of Artificial Sequence Synthetic polypeptide
DIQMTQITSSLSASLGDRVTIGCSASQGINNNLNWYQQKPDGTVKLLVYYTSSLHSGVPSRFSGSGSGTDYSLTISNLEPEDIATYYCQQFIKLPYTFGGGTKLEIK

SEQ ID NO: 46 - protein, 8 aa, Artificial Sequence
  Description of Artificial Sequence Synthetic peptide
QGINNNLN

SEQ ID NO: 47 - protein, 11 aa, Artificial Sequence
  Description of Artificial Sequence Synthetic peptide
LLVYYTSSLHS

SEQ ID NO: 48 - protein, 8 aa, Artificial Sequence
  Description of Artificial Sequence Synthetic peptide
QQFIKLPY

SEQ ID NO: 49 - protein, 116 aa, Artificial Sequence
  Description of Artificial Sequence Synthetic polypeptide
QVQLQQSGGELMKPGASVKLSCKATGYKFTGYWIEWVKQRPGHGLEWIGEILPGRGSTNYNEKFKGKATFTADTSSNTVYMQLSSLTTEDSAIYYCARGGRDFDFWGQGSTLTVSS

SEQ ID NO: 50 - protein, 9 aa, Artificial Sequence
  Description of Artificial Sequence Synthetic peptide
YKFTGYWIE

SEQ ID NO: 51 - protein, 14 aa, Artificial Sequence
  Description of Artificial Sequence Synthetic peptide
WIGEILPGRGSTNY

SEQ ID NO: 52 - protein, 8 aa, Artificial Sequence
  Description of Artificial Sequence Synthetic peptide
RGGRDFDF

SEQ ID NO: 53 - protein, 107 aa, Artificial Sequence
  Description of Artificial Sequence Synthetic polypeptide
DIQMTQTTSSLSASLGDRVTISCSASQGINSNLNWYQQKPDGTVKLLIYYTSSLHSGVPSRFSGSGSGTDYSLTISNLEPEDIATYYCQQYNKLPYTFGGGTKLEIK

SEQ ID NO: 54 - protein, 8 aa, Artificial Sequence
  Description of Artificial Sequence Synthetic peptide
QGINSNLN

SEQ ID NO: 55 - protein, 11 aa, Artificial Sequence
  Description of Artificial Sequence Synthetic peptide
LLIYYTSSLHS

SEQ ID NO: 56 - protein, 8 aa, Artificial Sequence
  Description of Artificial Sequence Synthetic peptide
QQYNKLPY

SEQ ID NO: 57 - protein, 119 aa, Artificial Sequence
  Description of Artificial Sequence Synthetic polypeptide
EVQLRQSGPVLVKPGASLKMSCKASGFTIADYYMTWVTQSHGQSLDWIGLINPYSGETTYNQKFKGKATLSVDKSSNTAYMDLNSLTSEDSAVYYCARWRLGYYAMDYWGRGTSVTVSS

SEQ ID NO: 58 - protein, 9 aa, Artificial Sequence
  Description of Artificial Sequence Synthetic peptide
FTIADYYMT

SEQ ID NO: 59 - protein, 14 aa, Artificial Sequence
  Description of Artificial Sequence Synthetic peptide
WIGLINPYSGETTY

SEQ ID NO: 60 - protein, 11 aa, Artificial Sequence
  Description of Artificial Sequence Synthetic peptide
RWRLGYYAMDY

SEQ ID NO: 61 - protein, 106 aa, Artificial Sequence
  Description of Artificial Sequence Synthetic polypeptide
ENVLTQSPAIMSASPGEKVTMTCSASSSVSYMHWYQQKSSTSPKVWIYDTSKLASGVPGRFSGSGSGNSYSLTISSMEAEDVATYYCFQGIGYPYTFGGGTKLEIK

SEQ ID NO: 62 - protein, 7 aa, Artificial Sequence
  Description of Artificial Sequence Synthetic peptide
SSVSYMH

SEQ ID NO: 63 - protein, 11 aa, Artificial Sequence
  Description of Artificial Sequence Synthetic peptide
VWIYDTSKLAS

SEQ ID NO: 64 - protein, 8 aa, Artificial Sequence
  Description of Artificial Sequence Synthetic peptide
FQGIGYPY

SEQ ID NO: 65 - protein, 116 aa, Artificial Sequence
  Description of Artificial Sequence Synthetic polypeptide
QVLLVQSGAEVKKPGASVKVSCKASGYTFTAYHIHWVRQAPGQGLEWMGIILPGRGSTTYAQKFQGRVTMTRDTSTSTVYMELSSLRSEDAAVYYCARGYRDFDYWGQGTLVTVST

SEQ ID NO: 66 - protein, 107 aa, Artificial Sequence
  Description of Artificial Sequence Synthetic polypeptide
EIVLTQSPATLSLSPGERATLSCKASQNVGTAVAWYQQKPGQAPRLLIYSASNRYTGIPARFSGSGSGTDFTLTISSLEPEDFAVYYCQQYTSYPRTFGGGTKVEIK

SEQ ID NO: 67 - protein, 116 aa, Artificial Sequence; identical to SEQ ID NO: 65
  Description of Artificial Sequence Synthetic polypeptide

SEQ ID NO: 68 - protein, 107 aa, Artificial Sequence
  Description of Artificial Sequence Synthetic polypeptide
EIVLTQSPATLSLSPGERATLSCKASQNVGTAVAWYQQKPGQTPRLLIYSASNRYTGIPARFSGSGSGTDFTLTISSLEPEDFAVYYCQQYTSYPRTFGQGTRLEIK

SEQ ID NO: 69 - protein, 116 aa, Artificial Sequence
  Description of Artificial Sequence Synthetic polypeptide
QVQLVQSGAEVRKPGASVRVSCKASGYTFTAYYMHWVRKAPGQGLEWLGRILPGRGSTMYAEKFQGRVTMTRDTSTSTDYMELSSLRSEDTAVYYCARGYRDFDYWGQGTLVTVSS

SEQ ID NO: 70 - protein, 107 aa, Artificial Sequence
  Description of Artificial Sequence Synthetic polypeptide
EKVMTQSPATLSVSPGERATLSCKASQNVGTAVAWYQQRPGQAPSLLIYSASNRYTGIPARFSGSGSGTEFTLTISSLQSEDFAVYYCQQYTSYPRTFGQGTRVDIK

SEQ ID NO: 71 - protein, 116 aa, Artificial Sequence
  Description of Artificial Sequence Synthetic polypeptide
QVQLVQSGPEVKKPGASVRVSCRASGYTFTAYYMHWVRQAPGQGLEWVGRILPGRGSTMYAEKFQGRVTLTRDTSTSTDYMDLSSLKSDDTAIYYCTRGYRDFDYWGQGTTVTVSS

SEQ ID NO: 72 - protein, 107 aa, Artificial Sequence; identical to SEQ ID NO: 66
  Description of Artificial Sequence Synthetic polypeptide

SEQ ID NO: 73 - protein, 116 aa, Artificial Sequence
  Description of Artificial Sequence Synthetic polypeptide
QMHLVQSGAEVKKPGASVKVSCKPSGYTFTAYYIHWLRQAPGQGLEWVGWILPGRGSTNYAQKFQGRVTMTRDTSISTAYMELASLRSDDTAVYYCARGYRDFDYWGQGTLVTISS

SEQ ID NO: 74 - protein, 107 aa, Artificial Sequence; identical to SEQ ID NO: 68
  Description of Artificial Sequence Synthetic polypeptide

SEQ ID NO: 75 - protein, 124 aa, Artificial Sequence
  Description of Artificial Sequence Synthetic polypeptide
QVQLLESGAEVKKPGASVKVSCKASGYPFTDYYMHWVRQAPGQGLEWMGWINPYNGDTTYAQKFQGRVTMTRDTSTSTVYMEVSSLRSDDTAVYYCARLRLLYDTSEYYVMDYWGQGTLVTVSS

SEQ ID NO: 76 - protein, 106 aa, Artificial Sequence
  Description of Artificial Sequence Synthetic polypeptide
DIQMTQSPSSLSASVGDRVTITCSASSSVNYMHWFQQKPGKAPKPLIYDTSKLASGVPPKFSGSGSGTDFTLTISSLQPDDFATYYCFQGSGYPYTFGRGTKLEIK

SEQ ID NO: 77 - protein, 124 aa, Artificial Sequence
  Description of Artificial Sequence Synthetic polypeptide
QVQLLASGAEVKKPGASVTVSCKASGYPFTDYYLHWVRQAPGQGLEWMGWINPYNGDTNYAPKFHGRVTMTRDTSISTAYMELSRLRSDDTAIYYCARLRLLYDTSEYYVMDYWGQGTTVTVSA

SEQ ID NO: 78 - protein, 106 aa, Artificial Sequence; identical to SEQ ID NO: 76
  Description of Artificial Sequence Synthetic polypeptide

SEQ ID NO: 79 - protein, 124 aa, Artificial Sequence
  Description of Artificial Sequence Synthetic polypeptide
QVQLLESGAEVKKPGASVKVSCKASGYPFTDYYLHWVRQAPGQGLEWMGWINPYNGDTNYAQKFQGRVTMTRDTSISTAYMELSRLRSDDTAVYYCARLRLLYDTSEYYVMDYWGQGTLVTVSS

SEQ ID NO: 80 - protein, 106 aa, Artificial Sequence; identical to SEQ ID NO: 76
  Description of Artificial Sequence Synthetic polypeptide

SEQ ID NO: 81 - protein, 124 aa, Artificial Sequence
  Description of Artificial Sequence Synthetic polypeptide
QVQLVQSGAEVKKPGASVKVSCKASGYPFTDYYIHWVRQAPGQGLQWMGWINPYNGDTNFAVKFQGWVTVTRDTSISTVYMGLRGLGSDDTAMYYCARLRLLYDTSEYYVMDYWGPGTTVTVSS

SEQ ID NO: 82 - protein, 106 aa, Artificial Sequence; identical to SEQ ID NO: 76
  Description of Artificial Sequence Synthetic polypeptide

SEQ ID NO: 83 - protein, 124 aa, Artificial Sequence
  Description of Artificial Sequence Synthetic polypeptide
EVQLVESGGGLVKPGESLRLSCAASGYPFTDYSMNWVRQAPGKGLEWISYINPYNGDIYDADSVKGRFTTSRDNARNSLYLQMNSLTAEDTAVYFCARLRLLYDTSEYYVMDYWGKGDLVTVSS

SEQ ID NO: 84 - protein, 106 aa, Artificial Sequence; identical to SEQ ID NO: 76
  Description of Artificial Sequence Synthetic polypeptide

SEQ ID NO: 85 - protein, 116 aa, Artificial Sequence
  Description of Artificial Sequence Synthetic polypeptide
QMQLVQSGSEVKKPGTSGKVSCKSSGYTFTAYAMQWVRQARGQGLEWIGRILPGRGSTNYAQIFRERVTITRNMSTSTSYMQLKSLRSEDTAVYYCAAGYRDFDYWGKGTVVTVSS

SEQ ID NO: 86 - protein, 107 aa, Artificial Sequence; identical to SEQ ID NO: 66
  Description of Artificial Sequence Synthetic polypeptide

SEQ ID NO: 87 - protein, 116 aa, Artificial Sequence
  Description of Artificial Sequence Synthetic polypeptide
QVQLVQSGAEVKKPGASVKLTCTASGYTFTAYYMHWMRQAPGQGLEWMGIVLPGRGSTNYAQKFQGRVTVTRDTSTNTVYMELNSLTSEDTAVYYCVRGYRDFDYWGPGTLVTVSS

SEQ ID NO: 88 - protein, 107 aa, Artificial Sequence
  Description of Artificial Sequence Synthetic polypeptide
ELVLTQSPATLSLSPGERATLSCKASQNVGTAVAWYQQKPGQAPRLLIYSASNRYTGIPARFSGSGSGTDFTLTVSSLEPEDFAVYYCQQYTSYPRTFGQGTKVEIK

SEQ ID NO: 89 - protein, 124 aa, Artificial Sequence; identical to SEQ ID NO: 77
  Description of Artificial Sequence Synthetic polypeptide

SEQ ID NO: 90 - protein, 106 aa, Artificial Sequence
  Description of Artificial Sequence Synthetic polypeptide
DIQMTQSPSRLSASIGDRVTITCSASSSVNYMHWYQQKPRKAPKLLIHDTSKLASDVPSRFSGGASGTEFTLTIDGLQPDDFATYFCFQGSGYPYTFGQGTNVEIQ

SEQ ID NO: 91 - protein, 124 aa, Artificial Sequence
  Description of Artificial Sequence Synthetic polypeptide
QVQLVQSGAEVKKPGASVKVSCKASGYPFTDYGISWVRQAPGQRLEWMGWINPYNGDTHYAQRFQGRVAMTTDTSMNTAYLELRSLKSDDAAVYYCVRLRLLYDTSEYYVMDYWGQGTLVTVSS

SEQ ID NO: 92 - protein, 106 aa, Artificial Sequence; identical to SEQ ID NO: 76
  Description of Artificial Sequence Synthetic polypeptide

SEQ ID NO: 93 - protein, 124 aa, Artificial Sequence
  Description of Artificial Sequence Synthetic polypeptide
AVQLVQSGAEVKKPGASVKVSCKASGYTFTDYYMNWVRQAPGQGLEWMGVFNPYNGDTRYTQKFKDRVTMTRDTSTSTVYMELSSLRSEDTAVYYCARLRLLYDTSEYYVMDYWGQGTTVTVSS

SEQ ID NO: 94 - protein, 106 aa, Artificial Sequence
  Description of Artificial Sequence Synthetic polypeptide
EIVLTQSPATLSLSPGERATLSCSASSSVNYMHWYQQKPGQAPRLLIYDTSKLASGIPARFSGSGSGTDFTLTISSLEPEDFAVYYCFQGSGYPYTFGGGTKVEIK

SEQ ID NO: 95 - protein, 124 aa, Artificial Sequence
  Description of Artificial Sequence Synthetic polypeptide
AVQLVQSGAEVKKPGASVKVSCKASGYTFTDYYMNWVRQAPGQGLEWMGVFNPYNGDTRYTQKFKDKVTMTIDKSTSTVYMELNSLRSEDTAVYYCARLRLLYDTSEYYVMDYWGQGTTVTVSS

SEQ ID NO: 96 - protein, 106 aa, Artificial Sequence; identical to SEQ ID NO: 94
  Description of Artificial Sequence Synthetic polypeptide

SEQ ID NO: 97 - protein, 124 aa, Artificial Sequence
  Description of Artificial Sequence Synthetic polypeptide
AVQLVQSGAEVKKPGASVKVSCKASGYTFTDYYMNWVRQAPGRGLEWMGVFNPYNGDTRYTQKFKDKVTLTIDKSTSTVYMELNSLRSEDTAVYYCARLRLLYDTSEYYVMDYWGQGTTVTVSS

SEQ ID NO: 98 - protein, 106 aa, Artificial Sequence; identical to SEQ ID NO: 94
  Description of Artificial Sequence Synthetic polypeptide

SEQ ID NO: 99 - protein, 124 aa, Artificial Sequence
  Description of Artificial Sequence Synthetic polypeptide
AVQLVQSGAEVKKPGASVKVSCKASGYTFTDYYMNWVKQAPGRGLEWMGVFNPYNGDTRYTQKFKDKATLTIDKSTSTVYMELNSLRSEDTAVYYCARLRLLYDTSEYYVMDYWGQGTTVTVSS

SEQ ID NO: 100 - protein, 106 aa, Artificial Sequence; identical to SEQ ID NO: 94
  Description of Artificial Sequence Synthetic polypeptide

SEQ ID NO: 101 - protein, 124 aa, Artificial Sequence; identical to SEQ ID NO: 93
  Description of Artificial Sequence Synthetic polypeptide

SEQ ID NO: 102 - protein, 106 aa, Artificial Sequence
  Description of Artificial Sequence Synthetic polypeptide
ENVLTQSPATLSLSPGERATLSCSASSSVNYMHWYQQKPGQAPRLLIYDTSKLASGIPARFSGSGSGNDFTLTISSLEPEDFAVYYCFQGSGYPYTFGGGTKVEIK

SEQ ID NO: 103 - protein, 124 aa, Artificial Sequence; identical to SEQ ID NO: 95
  Description of Artificial Sequence Synthetic polypeptide

SEQ ID NO: 104 - protein, 106 aa, Artificial Sequence; identical to SEQ ID NO: 102
  Description of Artificial Sequence Synthetic polypeptide

SEQ ID NO: 105 - protein, 124 aa, Artificial Sequence; identical to SEQ ID NO: 97
  Description of Artificial Sequence Synthetic polypeptide

SEQ ID NO: 106 - protein, 106 aa, Artificial Sequence; identical to SEQ ID NO: 102
  Description of Artificial Sequence Synthetic polypeptide

SEQ ID NO: 107 - protein, 124 aa, Artificial Sequence; identical to SEQ ID NO: 99
  Description of Artificial Sequence Synthetic polypeptide

SEQ ID NO: 108 - protein, 106 aa, Artificial Sequence; identical to SEQ ID NO: 102
  Description of Artificial Sequence Synthetic polypeptide

SEQ ID NO: 109 - protein, 124 aa, Artificial Sequence; identical to SEQ ID NO: 93
  Description of Artificial Sequence Synthetic polypeptide

SEQ ID NO: 110 - protein, 106 aa, Artificial Sequence
  Description of Artificial Sequence Synthetic polypeptide
ENVLTQSPATLSLSPGERATLSCSASSSVNYMHWYQQKPGQSPRLLIYDTSKLASGIPARFSGSGSGNDFTLTISSLEPEDFAVYYCFQGSGYPYTFGGGTKVEIK

SEQ ID NO: 111 - protein, 124 aa, Artificial Sequence; identical to SEQ ID NO: 95
  Description of Artificial Sequence Synthetic polypeptide

SEQ ID NO: 112 - protein, 106 aa, Artificial Sequence; identical to SEQ ID NO: 110
  Description of Artificial Sequence Synthetic polypeptide

SEQ ID NO: 113 - protein, 124 aa, Artificial Sequence; identical to SEQ ID NO: 97
  Description of Artificial Sequence Synthetic polypeptide

SEQ ID NO: 114 - protein, 106 aa, Artificial Sequence; identical to SEQ ID NO: 110
  Description of Artificial Sequence Synthetic polypeptide

SEQ ID NO: 115 - protein, 124 aa, Artificial Sequence; identical to SEQ ID NO: 99
  Description of Artificial Sequence Synthetic polypeptide

SEQ ID NO: 116 - protein, 106 aa, Artificial Sequence; identical to SEQ ID NO: 110
  Description of Artificial Sequence Synthetic polypeptide

SEQ ID NO: 117 - protein, 124 aa, Artificial Sequence
  Description of Artificial Sequence Synthetic polypeptide
QVQLVQSGAEVKKPGASVKVSCKASGYTFTDYYMNWVRQAPGQGLEWMGVFNPYNGDTRYTQKFKDRVTMTRDTSISTAYMELSRLRSDDTAVYYCARLRLLYDTSEYYVMDYWGQGTTVTVSS

SEQ ID NO: 118 - protein, 106 aa, Artificial Sequence; identical to SEQ ID NO: 94
  Description of Artificial Sequence Synthetic polypeptide

SEQ ID NO: 119 - protein, 124 aa, Artificial Sequence
  Description of Artificial Sequence Synthetic polypeptide
QVQLVQSGAEVKKPGASVKVSCKASGYTFTDYYMNWVRQAPGQGLEWMGVFNPYNGDTRYTQKFKDRVTMTIDKSISTAYMELSRLRSDDTAVYYCARLRLLYDTSEYYVMDYWGQGTTVTVSS

SEQ ID NO: 120 - protein, 106 aa, Artificial Sequence; identical to SEQ ID NO: 94
  Description of Artificial Sequence Synthetic polypeptide

SEQ ID NO: 121 - protein, 124 aa, Artificial Sequence
  Description of Artificial Sequence Synthetic polypeptide
QVQLVQSGAEVKKPGASVKVSCKASGYTFTDYYMNWVRQAPGRGLEWMGVFNPYNGDTRYTQKFKDRVTMTIDKSISTAYMELSRLRSDDTAVYYCARLRLLYDTSEYYVMDYWGQGTTVTVSS

SEQ ID NO: 122 - protein, 106 aa, Artificial Sequence; identical to SEQ ID NO: 94
  Description of Artificial Sequence Synthetic polypeptide

SEQ ID NO: 123 - protein, 124 aa, Artificial Sequence
  Description of Artificial Sequence Synthetic polypeptide
QVQLVQSGAEVKKPGASVKVSCKASGYTFTDYYMNWVRQAPGRGLEWIGVFNPYNGDTRYTQKFKDRATLTIDKSISTAYMELSRLRSDDTAVYYCARLRLLYDTSEYYVMDYWGQGTTVTVSS

SEQ ID NO: 124 - protein, 106 aa, Artificial Sequence; identical to SEQ ID NO: 94
  Description of Artificial Sequence Synthetic polypeptide

SEQ ID NO: 125 - protein, 124 aa, Artificial Sequence
  Description of Artificial Sequence Synthetic polypeptide
QVQLVQSGAEVKKPGASVKVSCKASGYPFTDYYMNWVRQAPGRGLEWIGVFNPYNGDTRYTQKFKDKATLTIDKSISTAYMELNRLRSDDTAVYYCARLRLLYDTSEYYVMDYWGQGTTVTVSS

SEQ ID NO: 126 - protein, 106 aa, Artificial Sequence; identical to SEQ ID NO: 94
  Description of Artificial Sequence Synthetic polypeptide

SEQ ID NO: 127 - protein, 124 aa, Artificial Sequence; identical to SEQ ID NO: 117
  Description of Artificial Sequence Synthetic polypeptide

SEQ ID NO: 128 - protein, 106 aa, Artificial Sequence
  Description of Artificial Sequence Synthetic polypeptide
ENVLTQSPATLSLSPGERATLSCSASSSVNYMHWYQQKPGQAPRLLIYDTSKLASGIPARFSGSGSGTDYTLTISSLEPEDFAVYYCFQGSGYPYTFGGGTKVEIK

SEQ ID NO: 129 - protein, 124 aa, Artificial Sequence; identical to SEQ ID NO: 119
  Description of Artificial Sequence Synthetic polypeptide

SEQ ID NO: 130 - protein, 106 aa, Artificial Sequence; identical to SEQ ID NO: 128
  Description of Artificial Sequence Synthetic polypeptide

SEQ ID NO: 131 - protein, 124 aa, Artificial Sequence; identical to SEQ ID NO: 121
  Description of Artificial Sequence Synthetic polypeptide

SEQ ID NO: 132 - protein, 106 aa, Artificial Sequence; identical to SEQ ID NO: 128
  Description of Artificial Sequence Synthetic polypeptide

SEQ ID NO: 133 - protein, 123 aa, Artificial Sequence
  Description of Artificial Sequence Synthetic polypeptide
QVQLVQSGAEVKKPGASVKVSCKASGYTFTDYYMNWVRQAPGRGLEWIGVFNPYNGDTRYTQKFKDRATLTIDKSISTAYMELSRLRSDDTAVYYCARLRLLYDTSEYYVMDYWGQGTTVTVS

SEQ ID NO: 134 - protein, 106 aa, Artificial Sequence; identical to SEQ ID NO: 128
  Description of Artificial Sequence Synthetic polypeptide

SEQ ID NO: 135 - protein, 124 aa, Artificial Sequence; identical to SEQ ID NO: 125
  Description of Artificial Sequence Synthetic polypeptide

SEQ ID NO: 136 - protein, 106 aa, Artificial Sequence; identical to SEQ ID NO: 128
  Description of Artificial Sequence Synthetic polypeptide

SEQ ID NO: 137 - protein, 124 aa, Artificial Sequence; identical to SEQ ID NO: 117
  Description of Artificial Sequence Synthetic polypeptide

SEQ ID NO: 138 - protein, 106 aa, Artificial Sequence
  Description of Artificial Sequence Synthetic polypeptide
ENVLTQSPATLSLSPGERATLSCSASSSVNYMHWYQQKPGQSPRLLIYDTSKLASGIPARFSGSGSGTDYTLTISSLEPEDFAVYFCFQGSGYPYTFGGGTKVEIK

SEQ ID NO: 139 - protein, 124 aa, Artificial Sequence; identical to SEQ ID NO: 119
  Description of Artificial Sequence Synthetic polypeptide

SEQ ID NO: 140 - protein, 106 aa, Artificial Sequence; identical to SEQ ID NO: 138
  Description of Artificial Sequence Synthetic polypeptide

SEQ ID NO: 141 - protein, 124 aa, Artificial Sequence; identical to SEQ ID NO: 121
  Description of Artificial Sequence Synthetic polypeptide

SEQ ID NO: 142 - protein, 106 aa, Artificial Sequence; identical to SEQ ID NO: 138
  Description of Artificial Sequence Synthetic polypeptide

SEQ ID NO: 143 - protein, 124 aa, Artificial Sequence; identical to SEQ ID NO: 123
  Description of Artificial Sequence Synthetic polypeptide

SEQ ID NO: 144 - protein, 106 aa, Artificial Sequence; identical to SEQ ID NO: 138
  Description of Artificial Sequence Synthetic polypeptide

SEQ ID NO: 145 - protein, 124 aa, Artificial Sequence; identical to SEQ ID NO: 125
  Description of Artificial Sequence Synthetic polypeptide

SEQ ID NO: 146 - protein, 106 aa, Artificial Sequence; identical to SEQ ID NO: 138
  Description of Artificial Sequence Synthetic polypeptide

SEQ ID NO: 147 - protein, 124 aa, Artificial Sequence
  Description of Artificial Sequence Synthetic polypeptide
AVQLQQSGPVRVKPGASVKMSCKASGYPFTDYYMNWVKQSHGRSLDWIGVFNPYQGDTRYTQKFKDKATLTIDKSSSTAYMELNSLTSEDSAVYYCARLRLLYDTSEYYVMDYWGQGTSVTVSS

SEQ ID NO: 148 - protein, 106 aa, Artificial Sequence; identical to SEQ ID NO: 5
  Description of Artificial Sequence Synthetic polypeptide

SEQ ID NO: 149 - protein, 124 aa, Artificial Sequence
  Description of Artificial Sequence Synthetic polypeptide
AVQLQQSGPVRVKPGASVKMSCKASGYPFTDYYMNWVKQSHGRSLDWIGVFNPYSGDTRYTQKFKDKATLTIDKSSSTAYMELNSLTSEDSAVYYCARLRLLYDTSEYYVMDYWGQGTSVTVSS

SEQ ID NO: 150 - protein, 106 aa, Artificial Sequence; identical to SEQ ID NO: 5
  Description of Artificial Sequence Synthetic polypeptide

SEQ ID NO: 151 - protein, 123 aa, Artificial Sequence
  Description of Artificial Sequence Synthetic polypeptide
AVQLQQSGPVRVKPGASVKMSCKASGYPFTDYYMNWVKQSHGRSLDWIGVFNPYNADTRYTQKFKDKATLTIDKSSSTAYMELNSLTSEDSAVYYCARLRLLYDTSEYYVMDYWGQGTSVTVS

SEQ ID NO: 152 - protein, 106 aa, Artificial Sequence; identical to SEQ ID NO: 5
  Description of Artificial Sequence Synthetic polypeptide

SEQ ID NO: 153 - protein, 118 aa, Artificial Sequence
  Description of Artificial Sequence Synthetic polypeptide
QVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSS

SEQ ID NO: 154 - protein, 9 aa, Artificial Sequence
  Description of Artificial Sequence Synthetic peptide
YTFTSFWMH

SEQ ID NO: 155 - protein, 14 aa, Artificial Sequence
  Description of Artificial Sequence Synthetic peptide
WMGRIDPNRGGTEY

SEQ ID NO: 156 - protein, 10 aa, Artificial Sequence
  Description of Artificial Sequence Synthetic peptide
GGNPYYPMDY

SEQ ID NO: 157 - protein, 107 aa, Artificial Sequence
  Description of Artificial Sequence Synthetic polypeptide
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIK

SEQ ID NO: 158 - protein, 8 aa, Artificial Sequence
  Description of Artificial Sequence Synthetic peptide
QGISNYLA

SEQ ID NO: 159 - protein, 11 aa, Artificial Sequence
  Description of Artificial Sequence Synthetic peptide
SLIYRANRLQS

SEQ ID NO: 160 - protein, 8 aa, Artificial Sequence
  Description of Artificial Sequence Synthetic peptide
LQYDDFPW

(19) United States
(12) Patent Application Publication
(10) Pub. No.: US 2024/0228658 A1
(43) Pub. Date: Jul. 11, 2024

(54) ANTI-5T4 ANTIBODIES AND USES THEREOF

(71) Applicant: IMMUNORIZON LTD., Rehovot (IL)

(72) Inventors: Oren BOGIN, Tel-Aviv (IL); Liat DASSA, Tel Aviv (IL)

(73) Assignee: IMMUNORIZON LTD., Rehovot (IL)

(21) Appl. No.: 18/558,673

(22) PCT Filed: May 2, 2022
(86) PCT No.: PCT/IL2022/050451

§ 371 Date: Nov. 2, 2023

Related U.S. Application Data
(60) Provisional application No. 63/183,636, filed on May 4, 2021.

(51) Int. Cl. C07K 16/30; A61K 39/00; A61P 35/00

(52) U.S. Cl. CPC C07K 16/30; A61P 35/00; A61K 2039/505

(57) ABSTRACT

The present disclosure describes a number of anti-5T4 antibodies. In some embodiments, anti-5T4 antibodies comprising certain complementarity determining regions (CDRs) are disclosed. The anti-5T4 antibodies disclosed herein can be used to treat various diseases such as cancer.

### CROSS REFERENCE TO RELATED APPLICATION(S)

This application is a 35 U.S.C. 371 National Phase Entry Application from PCT/IL2022/050451, filed on May 2, 2022, which claims the benefit of U.S. Provisional Application No. 63/183,636, filed on May 4, 2021, the disclosures of which are incorporated therein in their entirety by reference.

### SEQUENCE LISTING STATEMENT

The instant application contains a Sequence Listing which has been submitted electronically in ASCII format and is hereby incorporated by reference in its entirety. Said ASCII copy, created on Oct. 31, 2023, is named P-604133-USP_SQL and is 132,483 bytes in size.

### FIELD OF THE INVENTION

The present disclosure relates in general to antibodies. In one embodiment, the present disclosure describes the making and uses of anti-5T4 antibodies and anti-5T4 antigen binding fragments thereof.

### BACKGROUND

There has been considerable effort directed at the development of immunotherapeutic approaches for the treatment of cancer; many of which depend on targeting tumor-associated antigens (TAAs). While numerous TAAs have been identified, not all have the appropriate properties to enable safe and effective immune targeting. Such properties include a highly restricted expression profile on normal tissues but broad expression across many different cancer types. Furthermore, cell surface expression is an important property for antibody-targeted therapies.

5T4, also known as trophoblast glycoprotein, is a cell surface antigen that is rapidly internalized. 5T4 was discovered in the context of trying to identify shared cell surface molecules that may function to allow survival of the fetus as a semi-allograft in the mother, or a tumor in its host. Murine monoclonal antibodies were raised against purified glycoproteins from trophoblast membrane preparations from term human placenta and initially screened against different cancer cell lines and human peripheral blood mononuclear cells.

Expression of 5T4, as defined by immunohistochemistry, has been observed in a variety of solid tumors (i.e., lung, breast, ovarian, endometrial, bladder, pancreatic, esophageal, and gastric cancers), whereas expression in normal, adult tissues was found to be limited. 5T4 expression has been associated with advanced disease and/or worse clinical outcomes in patients with non-small-cell lung, colorectal, ovarian, or gastric cancer and pre-B acute lymphoblastic leukemia.

There is increasing evidence for key sub-populations of tumor-initiating cells reflecting normal tissue renewal properties retained and exploited for advantage by developing cancers. Poorly differentiated tumors in NSCLC have been associated with shorter patient survival and shorter time to recurrence following treatment. Using multiple experimental models with clinico-pathologic analysis of patient tumors to delineate a cellular hierarchy in NSCLC, it has been shown that 5T4 is expressed on tumor-initiating cells and associated with worse clinical outcome. Despite heterogeneous expression of 5T4 in NSCLC patient-derived xenografts, treatment with an anti-5T4 antibody-drug conjugate resulted in complete and sustained tumor regression. Thus, the aggressive growth of heterogeneous solid tumors can be blocked by therapeutic agents that target a 5T4 expressing subpopulation of cells.

5T4 molecules have been shown to be involved in the functional expression of CXCR4 at the cell surface in some embryonic and tumor cells. Both CXCL12 and CXCR4 expression have been associated with tumorigenesis in many cancers, and it is believed that CXCR4 expression facilitates the spread to tissues that highly express CXCL12 including lung, liver, lymph nodes and bone marrow. 5T4 is expressed by putative leukemia initiating cells in BCP-ALL, and these cells show the associated property of CXCL12/CXCR4 chemotaxis. 5T4-positive leukemia-initiating cells are likely attracted by CXCL12 produced by extramedullary sites where there is decreased therapeutic bioavailability leading to disease relapse following treatment.

Wnt protein intracellular signaling is a central component of many aspects of cellular regulation critical to normal development, homoeostasis and regeneration, while misregulation can lead to disease, including cancer. There are two pathways, the most characterized being the canonical Wnt/β-catenin pathway while non-canonical Wnt signaling through a cell autonomous planar cell polarity (PCP) type pathway can drive the modulation of actin and microtubular skeletons facilitating cell movement in development of cancer. 5T4 has been shown to interfere with Wnt/β-catenin signaling and concomitantly activate non-canonical Wnt pathways.

In view of the selective expression pattern of 5T4, and its association with a tumor-initiating phenotype plus a mechanistic involvement with cancer spread, several different immunotherapeutic strategies involving 5T4 have been developed. 5T4 vaccine, 5T4 antibody, 5T4 antibody-targeted superantigen, and 5T4 antibody-drug and 5T4 antibody-chimeric antigen receptors have all been explored for cancer treatment in preclinical and clinical studies. Although several “5T4-specific” antibodies made against specific peptides or sequences of the 5T4 molecule have been generated, their epitopes have often not been identified and could include parts of the 5T4 molecule containing leucine-rich repeats that are shared by a large number of proteins of diverse function and expression. Thus, there is a need to further develop anti-5T4 antibodies for immunotherapeutic uses.

### SUMMARY

In one embodiment, the present disclosure provides a number of anti-5T4 antibodies. In one embodiment, each of the anti-5T4 antibodies comprises three complementarity determining regions (CDRs) on a heavy chain (HCDR1, HCDR2, and HCDR3) and three CDRs on a light chain (LCDR1, LCDR2, and LCDR3), wherein
the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8; or
the HCDR1, HCDR2, and HCDR3 comprises the amino acid sequences of SEQ ID NOs: 10-12, and the LCDR1, LCDR2, and LCDR3 comprises the amino acid sequences of SEQ ID NOs: 14-16; or
the HCDR1, HCDR2, and HCDR3 comprises the amino acid sequences of SEQ ID NOs: 18-20, and the LCDR1, LCDR2, and LCDR3 comprises the amino acid sequences of SEQ ID NOs:22-24; or
the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32; or
the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40; or
the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48; or
the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56; or
the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

In one embodiment, each of the anti-5T4 antibodies comprises a heavy chain variable region and a light chain variable region, wherein the amino acid sequences for the heavy chain variable region and the light chain variable region can be one of the following pairs: SEQ ID NOs: 1 and 5; SEQ ID NOs:9 and 13; SEQ ID NOs: 17 and 21; SEQ ID NOs:25 and 29; SEQ ID NOs:33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs: 71-72; SEQ ID NOs:73-74; SEQ ID NOs:75-76; SEQ ID NOs:77-78; SEQ ID NOs:79-80; SEQ ID NOs:81-82; SEQ ID NOs:83-84; SEQ ID NOs:85-86; SEQ ID NOs:87-88; SEQ ID NOs:89-90; SEQ ID NOs:91-92; SEQ ID NOs:93-94; SEQ ID NOs:95-96; SEQ ID NOs:97-98; SEQ ID NOs:99-100; SEQ ID NOs:101-102; SEQ ID NOs:103-104; SEQ ID NOs:105-106; SEQ ID NOs:107-108; SEQ ID NOs: 109-110; SEQ ID NOs:111-112; SEQ ID NOs:113-114; SEQ ID NOs:115-116; SEQ ID NOs:117-118; SEQ ID NOs:119-120; SEQ ID NOs:121-122; SEQ ID NOs:123-124; SEQ ID NOs:125-126; SEQ ID NOs:127-128; SEQ ID NOs:129-130; SEQ ID NOs:131-132; SEQ ID NOs: 133-134; SEQ ID NOs:135-136; SEQ ID NOs:137-138; SEQ ID NOs:139-140; SEQ ID NOs: 141-142; SEQ ID NOs:143-144; SEQ ID NOs:145-146; SEQ ID NOs:147-148; SEQ ID NOs: 149-150; or SEQ ID NOs: 151-152.

In one embodiment, the present disclosure provides a composition comprising a pharmaceutically acceptable carrier and an anti-5T4 antibody disclosed herein.

The present disclosure also provides polynucleotide sequences encoding the anti-5T4 antibodies disclosed herein, as well as vectors and host cells comprising such polynucleotide sequences.

In one embodiment, the anti-5T4 antibodies disclosed herein can be used to treat diseases such as cancer, autoimmune diseases, GvHD, viral infection or bacterial infection.

These and other aspects of the anti-5T4 antibodies will be appreciated from the ensuing descriptions of the figures and detailed description of the anti-5T4 antibodies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the anti-5T4 antibodies and uses thereof are described herein, by way of example only, with reference to the accompanying drawings. With specific reference now to the drawings in detail, it is stressed that the particulars shown are by way of example and for purposes of illustrative discussion of embodiments of the anti-5T4 antibodies and uses thereof. In this regard, the description taken with the drawings makes apparent to those skilled in the art how embodiments of the anti-5T4 antibodies and uses thereof may be practiced.

FIGS. 1A-1D shows binding of recombinant human 5T4 ECD protein fused to human Fc using ELISA (FIGS. 1A-1B) and FACs (FIGS. 1C-1D). FIGS. 1A-1B show serum binding to recombinant human 5T4 ECD protein fused to human Fc by ELISA following subcutaneous (s.c.) immunization (FIG. 1A) or intraperitoneal (i.p) immunization (FIG. 1B).

FIGS. 1C-1D shows serum binding to CHO cells over expressing 5T4 by FACS (FIG. 1C) or CHO parental cells (FIG. 1D). TB2—test bleed 2. Reference numbers of different mice are provided. hIgG1—negative binding control. Anti-TPBG (anti-trophoblast glycoprotein) is used as a positive control.

FIG. 2 provides a table of the identities of selected mouse hybridoma clones.

FIGS. 3A-3B show SDS-PAGE of purified mAb clones under reducing conditions.

FIGS. 4A-4P show SEC-HPLC analysis of representative purified mAb clones 1, 2, 3, 4, 5, 6, 7, 8, 9, 10, 13, 15, 16, 17, 18, and 19.

FIGS. 5A-5D show ELISA binding curves of representative purified mAb clones.

FIGS. 6A-6H show FACS binding of representative purified mAb clones with CHO cells overexpressing human 5T4 (FIGS. 6A-6C), with CHO overexpressing cyno 5T4 (FIGS. 6D-6F), and with MCF-7 breast cancer cell line (FIGS. 6G-6H). Tab is a positive control antibody. mIgG (mouseIgG) is negative control mouse IgG. hIgG (human IgG) is a negative control human IgG. Anti-5T4 Ab is a positive control hIgG antibody.

FIGS. 7A-7G show Octet analysis of selected purified mAb, wherein FIGS. 7A-7F provides the Octet test analysis data of representative mAb clones, and FIG. 7G summarizes the ranked KD values.

FIG. 8 shows epitope binning analysis performed by ELISA identifying three major groups differentiated in their epitope. Group1 (circle continuous line), Group 2 (square dashed line), and Group 3 (circle dashed line).

FIGS. 9A-9C show binding of some embodiments of purified chimeric 5T4 mAbs by ELISA (FIG. 9A), by FACS with CHO cells (FIG. 9B) and by FACS with MCF-7 cells (FIG. 9C). Tab is a positive control antibody. hIgG is a negative control human-IgG.

FIGS. 10A-10B show an embodiment of a Tribody structure (FIG. 10A) and an embodiment of a ProTribody structure (FIG. 10B). VL—light chain variable region of anti CD3 Fab, VH—heavy chain variable region of anti CD3 Fab, HSA—human serum albumin, Anti-NK—anti natural killer cell antigen, anti-TAA—anti-tumor associated antigen. CD3CAP—a masking moiety that blocks the anti CD3 binding to Cd3 Antigen. Linkers (with or without protease cleavage) shown in these two embodiments, may be the same or different, or in some embodiments may not be present at any given position.

FIG. 11 shows embodiments of expressed Tribody antibody constructs analyzed by SDS-PAGE. See Table 1 for identification of SEQ ID NOs: for each construct. R-under reducing conditions. NR-under non-reducing conditions.

FIGS. 12A-12B show SDS-PAGE (FIG. 12A) and SEC-HPLC (FIG. 12B) analysis of Tribody antibody construct (IM-1222) including an anti-5T4 binding domain (5T4_IM53).

FIGS. 13A-13B show MS analysis of a Tribody construct (IM-1222) including an anti-5T4 binding domain (5T4_IM53), under reducing conditions (FIG. 13A) and intact (FIG. 13B).

FIGS. 14A-14B show SDS-PAGE (FIG. 14A) and SEC-HPLC (FIG. 14B) analysis of a Tribody antibody construct (IM-1178) including an anti-5T4 binding domain (5T4_IM24).

FIGS. 15A-15B show MS analysis of Tribody construct including an anti-5T4 binding domain (5T4_IM24), under reducing conditions (FIG. 15A) and intact (FIG. 15B).

FIGS. 16A-16H shows the results of ELISA screens for binding of different embodiments of purified humanized Tribody antibody constructs comprising different anti-5T4 binding domains.

FIGS. 17A-17O show FACS screen for binding of purified humanized Tribody constructs comprising different anti-5T4 binding domains to CHO cells over-expressing human 5T4 (FIGS. 17A-17H) or to NCI-H226 lung cancer cell line (FIGS. 17I-17O).

FIGS. 18A and 18B show in vitro cytotoxicity mediated by different embodiments of humanized Tribody constructs for NCI-H226 lung cancer cells (FIG. 18A) and MDA-MB-231 human breast adenocarcinoma cells (FIG. 18B). Tribody IM1222 comprises a 5T4_IM53 anti-5T4 binding domain. Tribody IM1062 comprises a positive control anti-5T4 binding domain, and TriBody IM1184 comprises a positive control anti-5T4 binding domain with CD3 CAP moiety.

FIG. 19 shows in vivo efficacy of Tribody constructs in xenograft mouse model. The Tribody constructs used each contain a single 5T4 binding domain.

### DETAILED DESCRIPTION

The present disclosure presents isolated anti-5T4 antibodies, wherein unique CDR sequences of anti-5T4 mAb are provided within a humanized framework (chimeric antibody; humanized antibody). In addition, incorporation of the 5T4 antigen binding regions of these anti-5T4 antibodies into multi-valent antibody construct is demonstrated. The anti-5T4 antibodies disclosed herein could potentially be used as an immunotherapeutic treatment for a medical condition, for example cancer.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind a 5T4 epitope when the equilibrium dissociation constant is ≤10⁻⁵, 10⁻⁶, or 10⁻⁷ M. In some embodiments, the equilibrium dissociation constant may be≤10⁻⁸ M or 10⁻⁹ M. In some further embodiments, the equilibrium dissociation constant may be≤10⁻¹⁰ M, 10⁻¹¹ M, or 10⁻¹²M. In some embodiments, the equilibrium dissociation constant may be in the range of ≤10⁻⁵ M to 10⁻¹²M.

Half maximal effective concentration (EC₅₀) refers to the concentration of a drug, antibody or toxicant which induces a response halfway between the baseline and maximum responses after a specified exposure time. In some embodiments, the response comprises a binding affinity. In some embodiments, the response comprises a functional response for example an agonistic response. A skilled artisan would appreciate that as used herein in certain embodiments, the EC₅₀ measurement of an anti-5T4 antibody disclosed herein provides a measure of a half-maximal binding of the anti-5T4 antibody to the 5T4 antigen (EC₅₀ binding). The skilled artisan would appreciate that as used herein in certain embodiments, the EC₅₀ measurement of an anti-5T4 antibody disclosed herein provides a measure of a half-maximal effective concentration of the anti-5T4 antibody to induce an agonist response (EC₅₀ functional agonism).

In some embodiments, EC₅₀ comprises the concentration of antibody required to obtain a 50% agonist response that would be observed upon antibody binding. In certain embodiments, a measure of EC₅₀ is commonly used as a measure of a drug's potency and may in some embodiments, reflect the binding of the antibody to the receptor. In some embodiments, anti-5T4 antibodies having nanomolar EC₅₀ binding concentration measurements comprise tight binding anti-5T4 antibodies. In some embodiments, anti-5T4 antibodies having nanomolar EC₅₀ functional agonism concentration measurements comprise functionally effective agonistic antibodies. In certain embodiments, an anti-5T4 antibody disclosed herein comprises a tight binder to the 5T4 molecule. In certain embodiments, an anti-5T4 antibody disclosed herein comprises an agonist for the 5T4 molecule. In certain embodiments, an anti-5T4 antibody disclosed herein comprises a tight binding agonist for the 5T4 molecule.

In some embodiments, the binding EC₅₀ of an anti-5T4 antibody is in the nanomolar range. In some embodiments, the binding EC₅₀ of an anti-5T4 antibody comprises a range of about 0.05-100 nM. In some embodiments, the binding EC₅₀ of an anti-5T4 antibody comprises a range of about 0.05-50 nM. In some embodiments, the binding binding EC₅₀ of an anti-5T4 antibody comprises a range of about 0.05-20 nM. In some embodiments, the binding EC₅₀ of an anti-5T4 antibody comprises a range of about 0.05-10 nM. In some embodiments, the binding EC₅₀ of an anti-5T4 antibody comprises a range of about 0.1-100 nM. In some embodiments, the binding EC₅₀ of an anti-5T4 antibody comprises a range of about 0.1-50 nM. In some embodiments, the binding EC₅₀ of an anti-5T4 antibody comprises a range of about 0.1-20 nM. In some embodiments, the binding EC₅₀ of an anti-5T4 antibody comprises a range of about 0.1-10 nM. In some embodiments, the binding EC₅₀ of an anti-5T4 antibody comprises a range of about 1-100 nM. In some embodiments, the binding EC₅₀ of an anti-5T4 antibody comprises a range of about 1-20 nM. In some embodiments, the binding EC₅₀ of an anti-5T4 antibody comprises a range of about 20-40 nM. In some embodiments, the binding EC₅₀ of an anti-5T4 antibody comprises a range of about 40-60 nM. In some embodiments, the binding EC₅₀ of an anti-5T4 antibody comprises a range of about 60-80 nM. In some embodiments, the binding EC₅₀ of an anti-5T4 antibody comprises a range of about 80-100 nM. In some embodiments, the binding EC₅₀ of an anti-5T4 antibody comprises a range of about 1-40 nM. In some embodiments, the binding EC₅₀ of an anti-5T4 antibody comprises a range of about 1-60 nM. In some embodiments, the binding EC₅₀ of an anti-5T4 antibody comprises a range of about 1-80 nM. In some embodiments, the binding EC₅₀ of an anti-5T4 antibody comprises a range of about 1-50 nM. In some embodiments, the binding EC₅₀ of an anti-5T4 antibody comprises a range of about 0.05-5 nM. In some embodiments, the binding EC₅₀ of an anti-5T4 antibody comprises a range of about 0.1-5 nM. In some embodiments, the binding EC₅₀ of an anti-5T4 antibody comprises a range of about 0.05-20 nM.

In some embodiments, the binding EC₅₀ of an anti-5T4 antibody comprises a range of about 0.05-5 nM. In some embodiments, the binding EC₅₀ of an anti-5T4 antibody comprises a range of about 0.1-5 nM. In some embodiments, the binding EC₅₀ of an anti-5T4 antibody comprises a range of about 1-5 nM. In some embodiments, the binding EC₅₀ of an anti-5T4 antibody comprises a range of about 0.05-10 nM. In some embodiments, the binding EC₅₀ of an anti-5T4 antibody comprises a range of about 0.1-10 nM. In some embodiments, the binding EC₅₀ of an anti-5T4 antibody comprises a range of about 1-10 nM. In some embodiments, the binding EC₅₀ of an anti-5T4 antibody comprises a range of about 5-10 nM. In some embodiments, the binding EC₅₀ of an anti-5T4 antibody comprises a range of about 0.05-15 nM. In some embodiments, the binding EC₅₀ of an anti-5T4 antibody comprises a range of about 0.01-15 nM. In some embodiments, the binding EC₅₀ of an anti-5T4 antibody comprises a range of about 1-15 nM.

In some embodiments, the EC₅₀ measuring functional agonism is referred herein as the function EC₅₀, having all the same qualities. In some embodiments, the functional EC₅₀ of an anti-5T4 antibody is in the nanomolar range. In some embodiments, the functional EC₅₀ of an anti-5T4 antibody comprises a range of about 0.05-100 nM. In some embodiments, the functional EC₅₀ of an anti-5T4 antibody comprises a range of about 0.05-50 nM. In some embodiments, the functional EC₅₀ of an anti-5T4 antibody comprises a range of about 0.05-20 nM. In some embodiments, the functional EC₅₀ of an anti-5T4 antibody comprises a range of about 0.05-10 nM. In some embodiments, the functional EC₅₀ of an anti-5T4 antibody comprises a range of about 0.1-100 nM. In some embodiments, the functional EC₅₀ of an anti-5T4 antibody comprises a range of about 0.1-50 nM. In some embodiments, the functional EC₅₀ of an anti-5T4 antibody comprises a range of about 0.1-20 nM. In some embodiments, the functional EC₅₀ of an anti-5T4 antibody comprises a range of about 0.1-10 nM. In some embodiments, the functional EC₅₀ of an anti-5T4 antibody comprises a range of about 1-100 nM. In some embodiments, the functional EC₅₀ of an anti-5T4 antibody comprises a range of about 1-20 nM. In some embodiments, the functional EC₅₀ of an anti-5T4 antibody comprises a range of about 20-40 nM. In some embodiments, the functional EC₅₀ of an anti-5T4 antibody comprises a range of about 40-60 nM. In some embodiments, the functional EC₅₀ of an anti-5T4 antibody comprises a range of about 60-80 nM. In some embodiments, the functional EC₅₀ of an anti-5T4 antibody comprises a range of about 80-100 nM. In some embodiments, the functional EC₅₀ of an anti-5T4 antibody comprises a range of about 1-40 nM. In some embodiments, the functional EC₅₀ of an anti-5T4 antibody comprises a range of about 1-60 nM. In some embodiments, the functional EC₅₀ of an anti-5T4 antibody comprises a range of about 1-80 nM. In some embodiments, the functional EC₅₀ of an anti-5T4 antibody comprises a range of about 1-50 nM. In some embodiments, the functional EC₅₀ of an anti-5T4 antibody comprises a range of about 0.05-5 nM. In some embodiments, the functional EC₅₀ of an anti-5T4 antibody comprises a range of about 0.1-5 nM. In some embodiments, the functional EC₅₀ of an anti-5T4 antibody comprises a range of about 0.05-20 nM.

In some embodiments, the functional EC₅₀ of an anti-5T4 antibody comprises a range of about 0.05-5 nM. In some embodiments, the functional EC₅₀ of an anti-5T4 antibody comprises a range of about 0.1-5 nM. In some embodiments, the functional EC₅₀ of an anti-5T4 antibody comprises a range of about 1-5 nM. In some embodiments, the functional EC₅₀ of an anti-5T4 antibody comprises a range of about 0.05-10 nM. In some embodiments, the functional EC₅₀ of an anti-5T4 antibody comprises a range of about 0.1-10 nM. In some embodiments, the functional EC₅₀ of an anti-5T4 antibody comprises a range of about 1-10 nM. In some embodiments, the functional EC₅₀ of an anti-5T4 antibody comprises a range of about 5-10 nM. In some embodiments, the functional EC₅₀ of an anti-5T4 antibody comprises a range of about 0.05-15 nM. In some embodiments, the functional EC₅₀ of an anti-5T4 antibody comprises a range of about 0.01-15 nM. In some embodiments, the functional EC₅₀ of an anti-5T4 antibody comprises a range of about 1-15 nM.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art. In some embodiments, an antibody disclosed herein comprises a precursor construct wherein the antigen binding site may be blocked by a regulatory domain, wherein exposure of the binding site comprise a regulated exposure based on environmental conditions, for example but not limited to exposure to a tumor micro-environment.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide. The linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)2 comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)2 fragments, scFv fragments, Fv fragments, minibodies, diabodies, triabodies, and tetrabodies.

In one embodiment, the anti-5T4 antibodies disclosed herein can be incorporated as part of a bispecific antibody. In one embodiment, the anti-5T4 antibodies disclosed herein can be incorporated as part of a multi-specific antibody. As it is generally known in the art, a bispecific antibody is a recombinant protein that includes antigen-binding fragments of two different monoclonal antibodies, and is thereby capable of binding two different antigens. In one embodiment, the anti-5T4 antibodies disclosed herein can be incorporated as part of a tri-specific antibody. In one embodiment, the anti-5T4 antibodies disclosed herein can be incorporated as part of a multi-specific antibody. Similarly, a multi-specific antibody is a recombinant protein that includes antigen-binding fragments of at least two different monoclonal antibodies, such as two, three or four different monoclonal antibodies.

In some embodiments, the anti-5T4 antibodies disclosed herein are bi-valent for 5T4. In some embodiments, the anti-5T4 antibodies disclosed herein are monovalent for binding 5T5.

In some embodiments, bispecific, tri-specific, or multi-specific antibodies are used for cancer immunotherapy by simultaneously targeting more than one antigen target, for example but not limited to, a cytotoxic T cell (CTL) as well as a tumor associated antigen (TAA), or simultaneously targeting more than one CTL, such as targeting a CTL receptor component such as CD3, an effector natural killer (NK) cells, and a tumor associated antigen (TAA), wherein for example the TAA comprises 5T4.

### Anti-5T4 Antibodies

The present disclosure provides a number of anti-5T4 antibodies. In one embodiment, each of the anti-5T4 antibodies comprises a set of three complementarity determining regions (CDRs) on a heavy chain (HCDR1, HCDR2, and HCDR3) and a set of three CDRs on a light chain (LCDR1, LCDR2, and LCDR3).

In one embodiment, the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8.

In one embodiment, the HCDR1, HCDR2, and HCDR3 comprises the amino acid sequences of SEQ ID NOs: 10⁻¹², and the LCDR1, LCDR2, and LCDR3 comprises the amino acid sequences of SEQ ID NOs: 14-16.

In one embodiment, the HCDR1, HCDR2, and HCDR3 comprises the amino acid sequences of SEQ ID NOs: 18-20, and the LCDR1, LCDR2, and LCDR3 comprises the amino acid sequences of SEQ ID NOs:22-24.

In one embodiment, the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32.

In one embodiment, the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40.

In one embodiment, the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48.

In one embodiment, the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56.

In one embodiment, the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

In one embodiment, an isolated anti-5T4 antibody comprising three complementarity determining regions (CDRs) on a heavy chain (HCDR1, HCDR2, and HCDR3) and three CDRs on a light chain (LCDR1, LCDR2, and LCDR3), comprises an antibody wherein
(i) the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8; or
(ii) the HCDR1, HCDR2, and HCDR3 comprises the amino acid sequences of SEQ ID NOs:10⁻¹², and the LCDR1, LCDR2, and LCDR3 comprises the amino acid sequences of SEQ ID NOs: 14-16; or
(iii) the HCDR1, HCDR2, and HCDR3 comprises the amino acid sequences of SEQ ID NOs:18-20, and the LCDR1, LCDR2, and LCDR3 comprises the amino acid sequences of SEQ ID NOs:22-24; or
(iv) the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32; or
(v) the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40; or
(vi) the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48; or
(vii) the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56; or
(viii) the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

In another embodiment, the anti-5T4 antibodies comprises heavy chain and light chain CDR sequences that are at least 80% (e.g., at least 85%, 90%, 95%, 96%, 97%, 98%, or 99%) identical to the amino acid sequences set forth above.

In one embodiment, each of the anti-5T4 antibodies presented herein comprises a heavy chain variable region (VH) and a light chain variable region (VL), wherein the amino acid sequences for the heavy chain variable region and the light chain variable region can be one of the following pairs: SEQ ID NOs: 1 and 5; SEQ ID NOs:9 and 13; SEQ ID NOs: 17 and 21; SEQ ID NOs:25 and 29; SEQ ID NOs: 33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs:71-72; SEQ ID NOs:73-74; SEQ ID NOs:75-76; SEQ ID NOs:77-78; SEQ ID NOs:79-80; SEQ ID NOs:81-82; SEQ ID NOs:83-84; SEQ ID NOs:85-86; SEQ ID NOs:87-88; SEQ ID NOs: 89-90; SEQ ID NOs:91-92; SEQ ID NOs:93-94; SEQ ID NOs:95-96; SEQ ID NOs:97-98; SEQ ID NOs:99-100; SEQ ID NOs: 101-102; SEQ ID NOs: 103-104; SEQ ID NOs: 105-106; SEQ ID NOs: 107-108; SEQ ID NOs: 109-110; SEQ ID NOs: 111-112; SEQ ID NOs:113-114; SEQ ID NOs: 115-116; SEQ ID NOs:117-118; SEQ ID NOs:119-120; SEQ ID NOs:121-122; SEQ ID NOs: 123-124; SEQ ID NOs:125-126; SEQ ID NOs:127-128; SEQ ID NOs:129-130; SEQ ID NOs: 131-132; SEQ ID NOs:133-134; SEQ ID NOs:135-136; SEQ ID NOs:137-138; SEQ ID NOs: 139-140; SEQ ID NOs: 141-142; SEQ ID NOs:143-144; SEQ ID NOs:145-146; SEQ ID NOs: 147-148; SEQ ID NOs: 149-150; or SEQ ID NOs: 151-152. In another embodiment, the anti-5T4 antibodies comprise VH and VL sequences that are at least 80% (e.g., at least 85%, 90%, 95%, 96%, 97%, 98%, or 99%) identical to the amino acid sequences set forth above.

In one embodiment, in view of the sequences for the heavy chain variable regions and light chain variable regions disclosed herein, one of ordinary skill in the art would readily employ standard techniques known in the art to construct an anti-5T4 scFv.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences that may include for example, but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to forms a diabody or a triabody.

In one embodiment, the anti-5T4 antibody presented herein can be an IgG, a Fv, a scFv, a Fab, a F(ab′)2, a minibody, a diabody, a triabody, a nanobody, a bispecific antibody, tri-specific, multi-specific, or a single domain antibody. For example, the anti-5T4 antibody can be IgG such as IgG1, IgG2, IgG3, or IgG4. In some embodiments, the anti-5T4 antibody comprises an IgG1. In some embodiments, the anti-5T4 antibody comprises an IgG2. In some embodiments, the anti-5T4 antibody comprises an IgG3. In some embodiments, the anti-5T4 antibody comprises an IgG4.

In one embodiment, the present disclosure provides antibodies that bind with high affinity to 5T4. In one embodiment, binding affinity is calculated by a modification of the Scatchard method as described by Frankel et al. (Mol. Immunol., 16:101-106, 1979). In another embodiment, binding affinity is measured by an antigen/antibody dissociation rate. In another embodiment, binding affinity is measured by a competition radioimmunoassay. In another embodiment, binding affinity is measured by ELISA. In another embodiment, antibody affinity is measured by flow cytometry.

In one embodiment, the present disclosure also provides isolated polynucleotide sequence encoding the heavy chain and light chain CDRs as described herein. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

In one embodiment, the present disclosure also provides isolated polynucleotide sequence encoding the heavy chain and light chain variable regions described herein. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

### Compositions for Use

In one embodiment, the present disclosure also provides a composition comprising the anti-5T4 antibody disclosed herein and a pharmaceutically acceptable carrier. Pharmaceutically acceptable carriers of use are well-known in the art. For example, Remington's Pharmaceutical Sciences, by E. W. Martin, Mack Publishing Co., Easton, PA, 15th Edition, 1975, describes compositions and formulations suitable for pharmaceutical delivery of the antibodies disclosed herein. In one embodiment, the composition comprises anti-5T4 antibodies that comprise a set of three complementarity determining regions (CDRs) on a heavy chain (HCDR1, HCDR2, and HCDR3) and a set of three CDRs on a light chain (LCDR1, LCDR2, and LCDR3).

In some embodiments of compositions, the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8.

In some embodiments of compositions, the HCDR1, HCDR2, and HCDR3 comprises the amino acid sequences of SEQ ID NOs: 10⁻¹², and the LCDR1, LCDR2, and LCDR3 comprises the amino acid sequences of SEQ ID NOs: 14-16.

In some embodiments of compositions, the HCDR1, HCDR2, and HCDR3 comprises the amino acid sequences of SEQ ID NOs: 18-20, and the LCDR1, LCDR2, and LCDR3 comprises the amino acid sequences of SEQ ID NOs:22-24.

In some embodiments of compositions, the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32.

In some embodiments of compositions, the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40.

In some embodiments of compositions, the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48.

In some embodiments of compositions, the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56.

In some embodiments of compositions, the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

In other embodiments, the composition comprises anti-5T4 antibodies having heavy chain and light chain CDR sequences that are at least 80% (e.g., at least 85%, 90%, 95%, 96%, 97%, 98%, or 99%) identical to the amino acid sequences set forth above.

In another embodiment, the composition comprises anti-5T4 antibodies having one of the following pairs of heavy chain variable region and light chain variable region: SEQ ID NOs: 1 and 5; SEQ ID NOs:9 and 13; SEQ ID NOs: 17 and 21; SEQ ID NOs:25 and 29; SEQ ID NOs:33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs:71-72; SEQ ID NOs:73-74; SEQ ID NOs:75-76; SEQ ID NOs:77-78; SEQ ID NOs:79-80; SEQ ID NOs:81-82; SEQ ID NOs:83-84; SEQ ID NOs:85-86; SEQ ID NOs:87-88; SEQ ID NOs:89-90; SEQ ID NOs:91-92; SEQ ID NOs:93-94; SEQ ID NOs:95-96; SEQ ID NOs:97-98; SEQ ID NOs:99-100; SEQ ID NOs: 101-102; SEQ ID NOs: 103-104; SEQ ID NOs: 105-106; SEQ ID NOs: 107-108; SEQ ID NOs: 109-110; SEQ ID NOs: 111-112; SEQ ID NOs: 113-114; SEQ ID NOs: 115-116; SEQ ID NOs: 117-118; SEQ ID NOs: 119-120; SEQ ID NOs: 121-122; SEQ ID NOs: 123-124; SEQ ID NOs: 125-126; SEQ ID NOs: 127-128; SEQ ID NOs:129-130; SEQ ID NOs:131-132; SEQ ID NOs:133-134; SEQ ID NOs: 135-136; SEQ ID NOs:137-138; SEQ ID NOs:139-140; SEQ ID NOs:141-142; SEQ ID NOs: 143-144; SEQ ID NOs: 145-146; SEQ ID NOs: 147-148; SEQ ID NOs: 149-150; or SEQ ID NOs:151-152. In another embodiment, the composition comprises anti-5T4 antibodies having VH and VL sequences that are at least 80% (e.g., at least 85%, 90%, 95%, 96%, 97%, 98%, or 99%) identical to the amino acid sequences set forth above.

In some embodiments of compositions, the antibodies disclosed herein can be in the form of a conjugate. As used herein, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate can also be referred to as an “immunoconjugate.” When the conjugate comprises an antibody linked to a drug (e.g., a cytotoxic agent), the conjugate can be referred to as an “antibody-drug conjugate”. Other antibody conjugates include, for example, multi-specific (such as bispecific or trispecific) antibodies and chimeric antigen receptors (CARs).

A composition comprising the anti-5T4 antibody or an antigen-binding fragment thereof can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The pharmaceutical compositions comprising the antibodies or antigen-binding fragments thereof disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Alternatively, administration can be intratumoral, e.g., local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

### Methods of Use

In some embodiments, the anti-5T4 antibodies disclosed herein can be used to treat a disease or condition. In some embodiments, the anti-5T4 antibodies disclosed herein can be used to treat diseases such as cancer. In some embodiments, the anti-5T4 antibodies disclosed herein can be used as a component of a vaccine. In some embodiments, the anti-5T4 antibodies disclosed herein can be used as part of an antibody-drug conjugate (ADC). In some embodiments, an anti-5T4 antibody disclosed herein can be used in methods of treating cancer, for example but not limited to treating non-small-cell lung carcinoma (NSCLC), breast cancer, mesothelioma, pancreatic cancer, renal cancer, prostate cancer, ovarian cancer, or colon cancer.

In some embodiments, the anti-5T4 antibodies disclosed herein can be used to treat a disease associated with 5T4. In some embodiments, the anti-5T4 antibodies disclosed herein can be used to treat a disease associated with over-expression of 5T4.

In some embodiments, the anti-5T4 antibodies disclosed herein comprise cytotoxic activities. In some embodiments, the anti-5T4 antibodies disclosed herein are cytotoxic to cancer or tumor cells.

In some embodiments, the anti-5T4 antibodies disclosed herein may be used in a method to a cancer or tumor. In some embodiments, the cancer or tumor comprises a solid cancer or tumor. In some embodiments, the cancer or tumor comprises a non-solid (diffuse) cancer or tumor. In some embodiments, the cancer or tumor comprises a metastasis of a cancer or tumor.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present anti-5T4 antibodies is provided. The terms “non-human animals” and “non-human mammals” are used interchangeably herein and include all vertebrates, e.g., mammals, such as non-human primates (e.g., higher primates), sheep, dog, rodent (e.g. mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%.

In one embodiment, the method comprises the step of administering to the subject a composition comprising an effective amount of the anti-5T4 antibody disclosed herein. In one embodiment, the composition comprises anti-5T4 antibodies having the heavy chain and light chain CDR sequences as described herein. In another embodiment, the composition comprises anti-5T4 antibodies having the VH and VL sequences as described herein.

One skilled in the art would appreciate that in some embodiments, modulation of an immune response encompasses a reduction of inflammation or elimination of inflammation in a situation wherein the expected outcome without the use of an anti-5T4 antibody described herein, would have been inflammation. One skilled in the art would appreciate that in some embodiments, treating a tumor or cancer encompasses a reduction of tumor size, growth, and or spread of the tumor or cancer, compared with the outcome without the use of an anti-5T4 antibody described herein.

In one embodiment, the present disclosure provides a method of treating a disease in a subject, comprising the step of administering to the subject a composition comprising an effective amount of the anti-5T4 antibody disclosed herein. In one embodiment, the composition comprises anti-5T4 antibodies having the heavy chain and light chain CDR sequences as described herein. In another embodiment, the composition comprises anti-5T4 antibodies having the VH and VL sequences as described herein.

In one embodiment, the present disclosure also provides uses of a composition comprising anti-5T4 antibodies for treating a disease in a subject. In one embodiment, the composition comprises anti-5T4 antibodies having the heavy chain and light chain CDR sequences as described herein. In another embodiment, the composition comprises anti-5T4 antibodies having the VH and VL sequences as described herein.

In one embodiment, the exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In another embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In another embodiment, the disease is an autoimmune disease that can be, but is not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopeniarpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, post-myocardial infarction syndrome, post-pericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenia purpura, type 1 diabetes, ulcerative colitis, uveitis, vasculitis, and vitiligo.

In some embodiments, the disease is a transplantation-related diseases such as graft-versus-host disease (GvHD). According to one embodiment, the GVHD is acute GVHD. According to another embodiment, the GVHD is chronic GVHD.

In another embodiment, the present disclosure provides a method of using a polynucleotide to treat a disease or condition as described above, wherein the polynucleotide encodes an anti-5T4 antibody as described herein.

As used herein, the terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an antibody” or “at least one antibody” may include a plurality of antibodies.

Throughout this application, various embodiments of the present disclosure may be presented in a range format. It should be understood that the description in range format is merely for convenience and brevity and should not be construed as an inflexible limitation on the scope of the anti-5T4 antibodies and uses thereof. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of +10% from the indicated number or range of numbers. In another embodiment, the term “about” refers to a deviance of +5% from the indicated number or range of numbers.

Unless otherwise defined, all technical and/or scientific terms used herein have the same meaning as commonly understood by one of ordinary skill in the art to which the anti-5T4 antibodies and uses thereof pertains. Although methods and materials similar or equivalent to those described herein can be used in the practice or testing of embodiments of the anti-5T4 antibodies and uses thereof, methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of making and using the anti-5T4 antibodies and variations thereof are described. This description is not intended to be limiting and changes in the components, sequence of steps, and other variations would be understood to be within the scope of the present anti-5T4 antibodies and uses thereof.

It is appreciated that certain features of the anti-5T4 antibodies and uses thereof, which are, for clarity, described in the context of separate embodiments, may also be provided in combination in a single embodiment. Conversely, various features of the anti-5T4 antibodies and uses thereof, which are, for brevity, described in the context of a single embodiment, may also be provided separately or in any suitable subcombination or as suitable in any other described embodiment of the anti-5T4 antibodies and uses thereof. Certain features described in the context of various embodiments are not to be considered essential features of those embodiments, unless the embodiment is inoperative without those elements.

Various embodiments and aspects of the present anti-5T4 antibodies as delineated hereinabove and as claimed in the claims section below find experimental support in the following examples.

### EXAMPLES

### Example 1

### Mouse Hybridoma Monoclonal Antibody Generation for 5T4

Objective: To generate monoclonal antibodies against 5T4 using mouse hybridoma technology.

Methods: Generation of Mouse Hybridoma Anti 5T4 Antibodies. Anti-5T4 antibodies were developed by immunizing i.p/s.c SJL mice with 5T4-ECD-hFc. The animals were bled and tested for antibody titer with ELISA (test-bleed 1 and test-bleed −2). Spleen cells from immunized mice with high titer were isolated and fused by standard fusion procedures to create hybridoma producing specific antibodies. Supernatants containing antibodies produced by pools of these cells were primary screened by ELISA for reactivity with 5T4-ECD protein fused to human Fc (5T4-ECD-hFc) and a secondary screening by FACS for reactivity with 5T4 protein over-expressing cells. For screening, large numbers of hybridoma supernatants for target antibody activity by FACS were tested.

The target over-expressing cells were placed into 96-well round-bottom polystyrene plates and incubated with neat supernatants from the hybridoma cultures. The cells were then washed, incubated with a fluorescence labeled secondary antibody. As a negative reference, non-target protein gene transfected parental cells were employed to test the supernatants (and found to be negative) to confirm that the reactive antibody recognized target protein specifically. Positive pools were identified and cloned by limiting dilution. After 1-2 fusions, positive clones producing specific antibodies were identified and selected by ELISA and FACS.

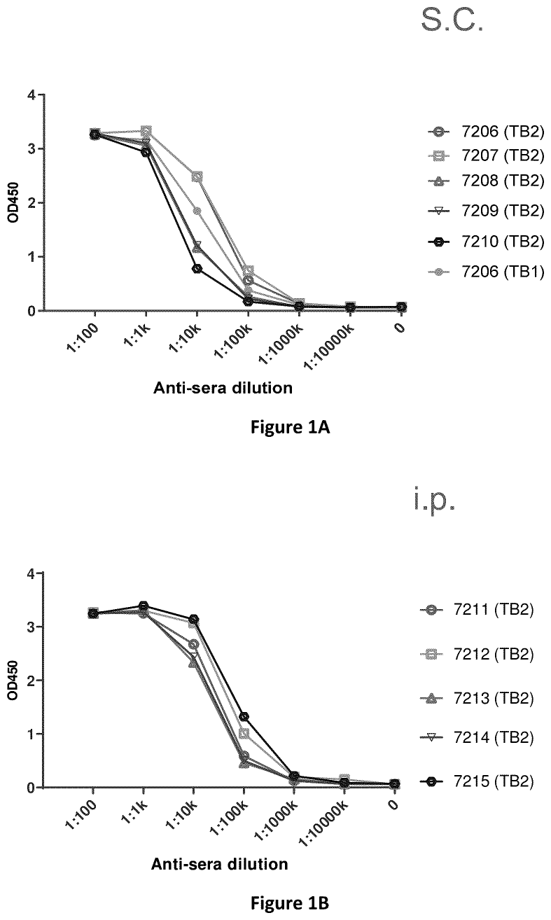

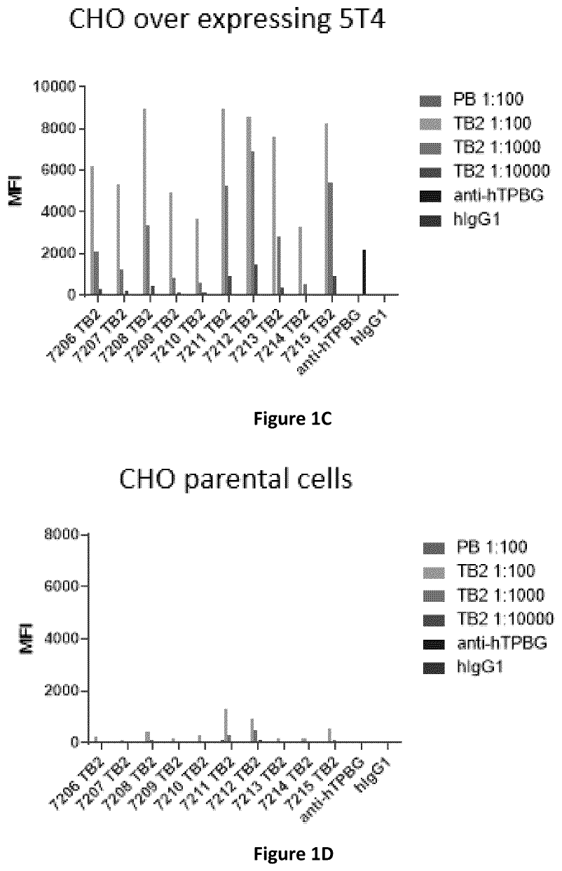

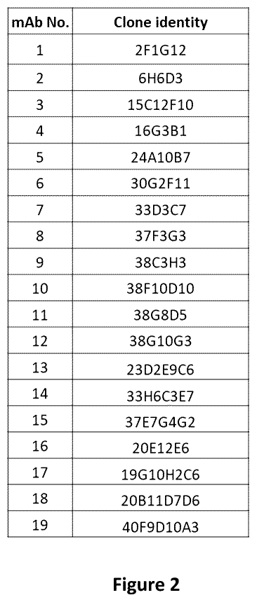

Results: Following animals' immunization, serum was tested for binding to recombinant human 5T4 ECD protein fused to human Fc by ELISA and by FACS. As shown in FIGS. 1A-1B, test bleed 2 of animals immunized s.c. (FIG. 1A) or i.p (FIG. 1B) in various dilutions showed positive titer against the human 5T4 by ELISA as compared to the pre-bleed samples. FIGS. 1C-1D demonstrate serial dilution of test bleed 2 positive titer against the human-5T4 over expressed in CHO cells (FIG. 1C) while no binding is observed on the CHO parental cells (FIG. 1D), indicating for 5T4 specificity. Following test-bleed analysis animals SJL #7211 and #7215 were selected for fusion. 96 well plates for primary screen and 24 well plates for secondary screen were obtained following electrofusion to identify positive clones by ELISA against recombinant human 5T4-ECD-Fc, with human Fc served as a negative control, and against CHO cells over expressing human-5T4 or cyno-5T4 to identify cross reactive clones, with CHO parental cells served as negative control. In the primary screen 200 clones were selected for secondary screening. Secondary screen revealed 30 clones that were further subcloned, and 19 clones were proceeded for 200 ml expression and protein A purification. FIG. 2 present the identity of the selected clones. A full listing of the 5T4 antibodies and FIG. 2 present the identity of the selected clones. A listing of the 5T4 antibodies and components thereof, disclosed herein, is provided below in Table 1, along with the corresponding SEQ ID NOs.

Summary: Mouse monoclonal hybridoma generation against human 5T4 protein, yielded 19 clones that were identified following primary and secondary screening by ELISA and FACS to be specific binders to human and cyno 5T4. These clones were further expressed purified and analyzed by SDS-PAGE and by SEC-HPLC and characterized for binding efficacy.

### Example 2

### Binding Characterization of the Mouse Hybridoma Monoclonal Antibodies Against Human-5T4

Objective: To express purify and characterize mouse monoclonal Abs against 5T4 by binding.

Methods: Expression and Purification of The Hybridoma Clones. Briefly, about (0.25-0.5)×10⁷ cells were inoculated into a roller bottle pre-filled with 100 mL antibody production medium (Hybridoma-SFM+2.5% FBS (Low IgG), and incubated in roller culture apparatus at 300 r/h speed for 14-16 days at 37° C., no CO2 condition. Thereafter, the cell suspension was transferred into a 350 mL centrifuge bottle and centrifuged at 3,220 g, 4° C., for 15 min, and then filtered with a 0.45 μm filtration capsule to remove the cells and cell debris. Then the culture supernatant was loaded onto a pre-equilibrated Protein A affinity column for affinity purification. Antibody was eluted from the column with 5 CV of elution buffer (0.1 M citrate sodium buffer, pH 3.0), and neutralized to final pH 7.0 with Trizma base, and then dialyzed against 100-fold of elution volume of PBS, pH 7.4, at 2-8° C. overnight, and sterile filtered with a 0.22 μm syringe filter in a biological safety cabinet. The purified antibody was then aliquoted and stored at −20° C. or −80° C. until use.

ELISA Binding to The Target Protein. Briefly, dilute target protein h5T4-His (cat #19845-H08H, supplier Sino Biological) into PBS with final concentration of 0.3 μg/mL h5T4-His, and coat 100 μL/well on ELISA plate (cat: 9018, supplier Corning) respectively. Incubate O/N, 4° C. The plates were blocked with 250 μL 1% BSA in PBST for 1 hr at 37° C. Wash four times with PBST. All the washes are done using Biotek (Elx 405). All the Abs were serial diluted and 100 μL/well diluted antibody were added to plate, incubate for 1 hr at 37° C. Wash 4 times with PBST. Add 100 μL/well goat Anti-Mouse IgG (Fab specific)-HRP, (SIGMA A3682) 1:10000, incubate for 0.5 hr at 37° C. Wash 4 times with PBST. 100 μL/well of TMB substrate was added and incubated at room temperature for 5 min. 100 u L/well of 1N HCl to terminate reaction. Plates were read using ELISA plate reader at 450 nm wavelength (instrument SpectraMax M5e). Data Analysis was performed using Graphpad prism 5 software by using nonlinear regression (curve fit): log (agonist) vs. response, agonist is antibody concentration (nM) and response is OD value.

FACS Binding to Cells. Briefly, directly harvest suspension cultured cells or TrypLE Express Enzyme (cat: 12604-013, supplier Life technologies) digest adherent cells before harvesting. Centrifuging at 1000 rpm for 5 min and discard the supernatant. Cells are suspended at a concentration of 2×10⁶/mL in FACS buffer (2% FBS in PBS) and add 100 μL/well of cell suspension to the plate (cat #3799, supplier Corning). Centrifuge the plates at 2000 rpm for 5 min, and discard the supernatant. Re-suspend the cells in 100 μL/well of Tribody set antibodies (400 nM start, 4-fold dilution, 8 point including 0 point) and incubate the plate for 60 min at 4° C. Centrifuge the plate at 2000 rpm, 4° C. for 5 min and discard supernatant. Then wash the cells 3 times with 170 μL FACS buffer. Re-suspend the cells at 100 μL/well with secondary antibody and incubate the plate for 30 min at 4° C. in dark. Centrifuge the plate at 2000 rpm, 4° C. for 5 min and discard supernatant. Then wash the cells 3 times with FACS buffer and analyze the sample with FACS verse.

Binding Affinities by Octet Test. Briefly, mAb were analyzed by Octet RED 384 instrument, where human 5T4-ECD Fc antigen at 200 nM and 100 nM served as the analyte and the purified mAbs at 5 μg/ml served as the ligand, bound to AMC sensor (anti mouse Fc antibody) with immobilized phase of 180 s, association phase of 300 s, dissociation phase of 180 s.

Epitope Binning by ELISA. Briefly, dilute target antibodies of human 5T4 into PBS with final concentration of 1 μg/ml and coat 100 μL/well on ELISA plate (cat: 9018, supplier Corning). Incubate O/N, 4° C. After blocking with 250 μL 1% BSA in PBST for 1 hr at 37° C., add series concentration of biotinylated antigen h5T4-His (lot: 1906191701, CP). Wash the plate 3 times with PBST after incubating for 1.5 hrs at 37° C., then add 100 μL of streptavidin-HRP (cat: S5512, supplier Sigma, 1:10000) to each well. After incubating for 1 hr at 37° C., wash the plate 4 times with PBST. 100 μL/well of TMB substrate was added and incubated at room temperature for 5 min. 100 μL/well of 1N HCL to terminate reaction. Plates were read using ELISA plate reader at 450 nm wavelength. Find EC80 of antigen using Graphpad prism 5 software.

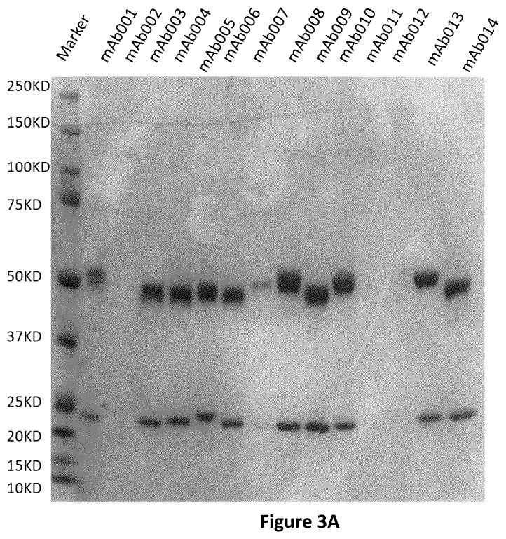

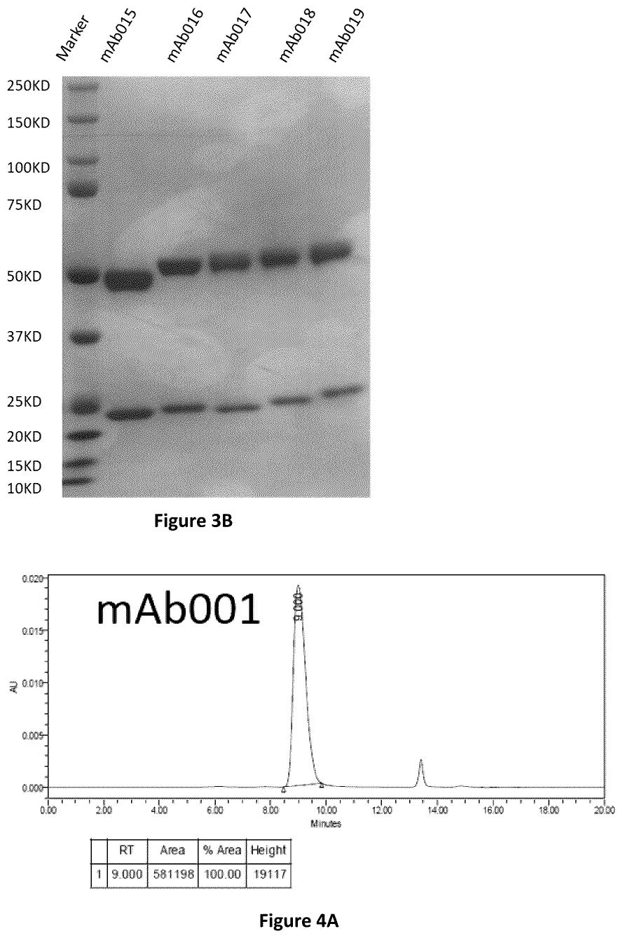

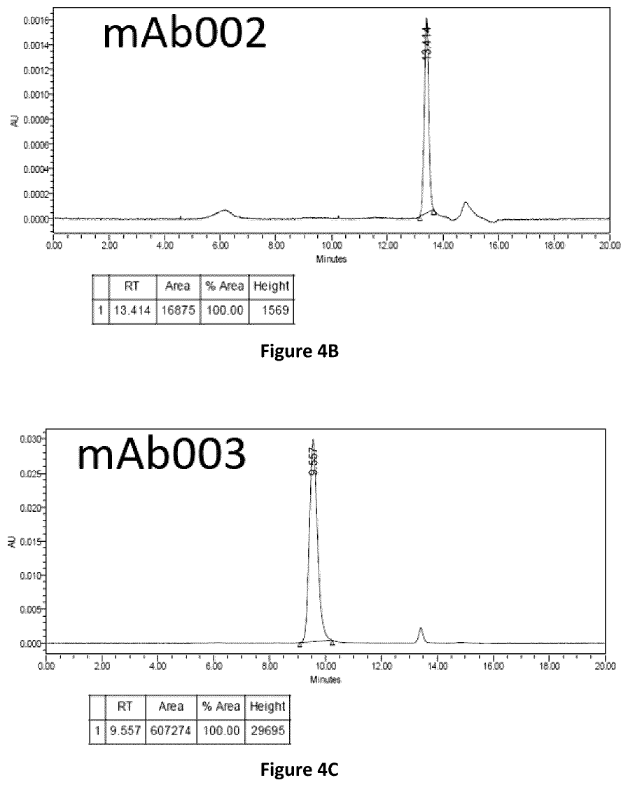

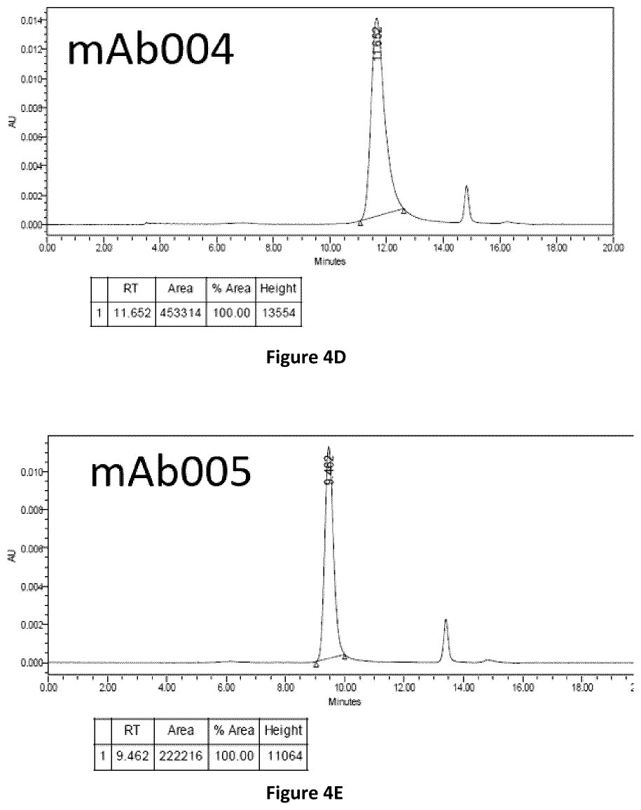

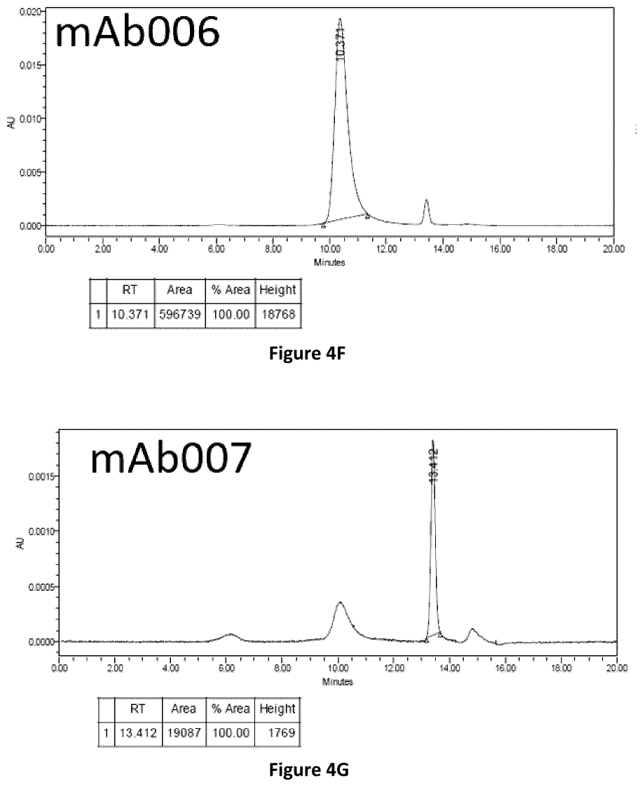

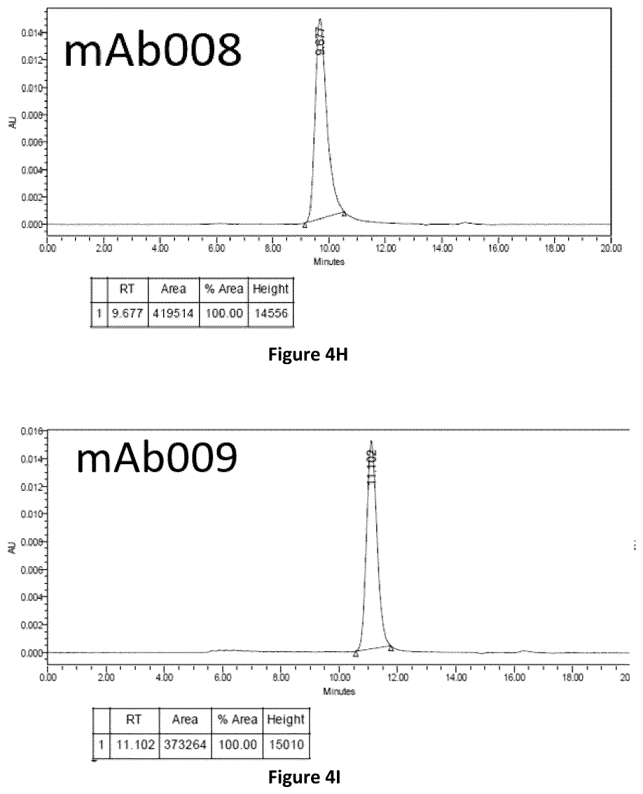

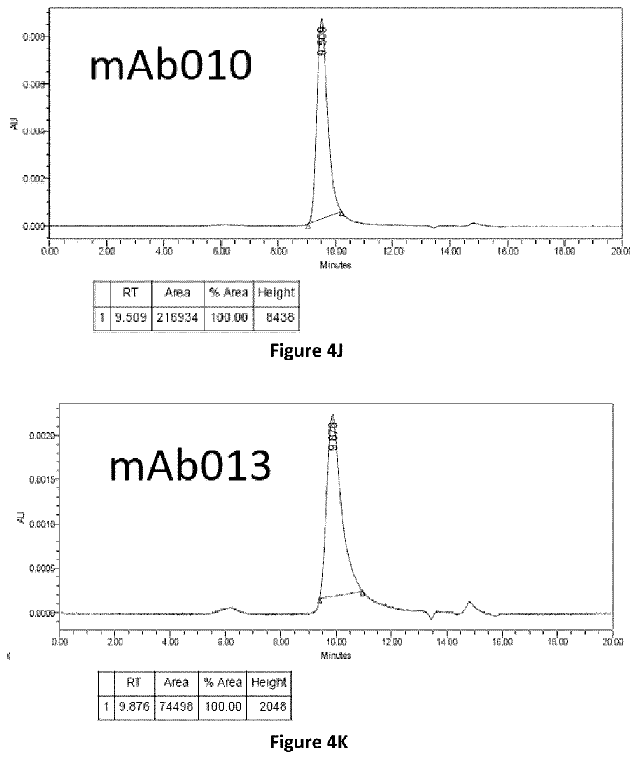

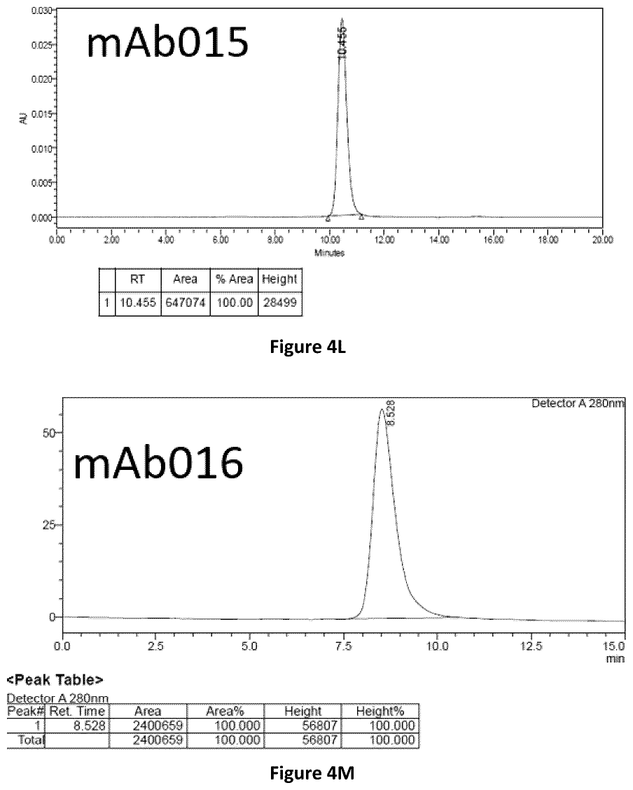

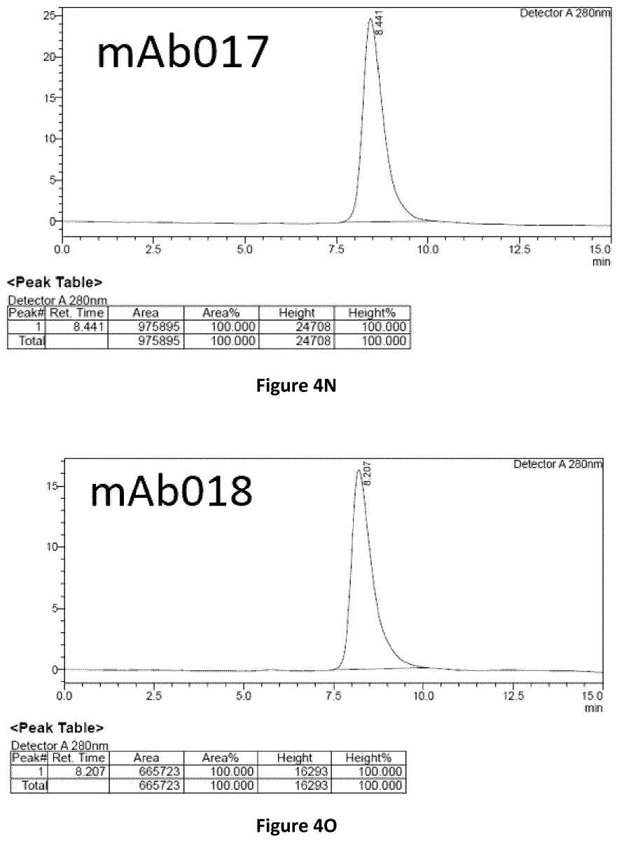

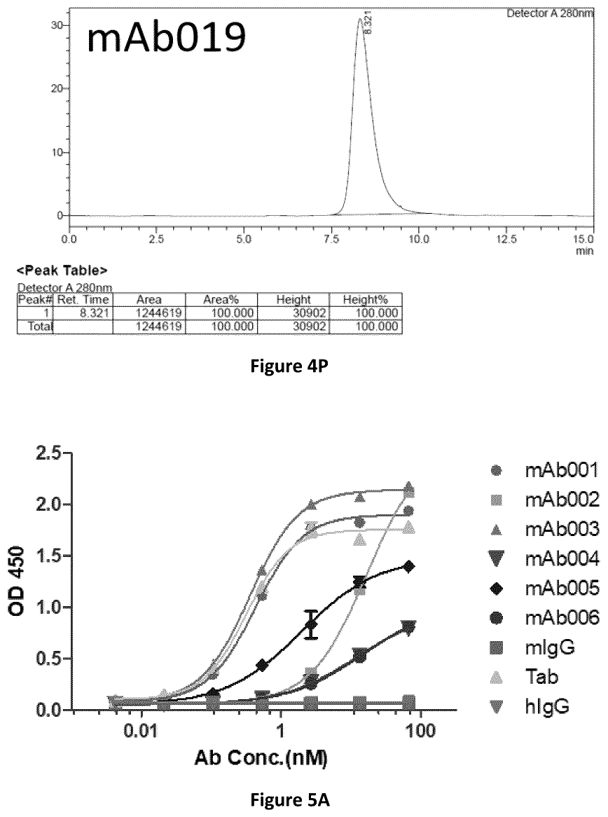

Results: FIGS. 3A-3B demonstrate SDS-PAGE analysis in reducing conditions of the 19 selected purified antibodies, indicating for 16 well-produced clones with two bands for each at the expected band size of the HC and the LC (50 kDa and 25 kDa, respectively). FIGS. 4A-4P present SEC-HPLC analysis of the selected purified clones, indicating a single sharp and uniform peak at retention time ranges from ˜8 to ˜13 min in SEC-HPLC. These results are in agreement with the expected retention time of the expected Mw based on mass calibration curve.

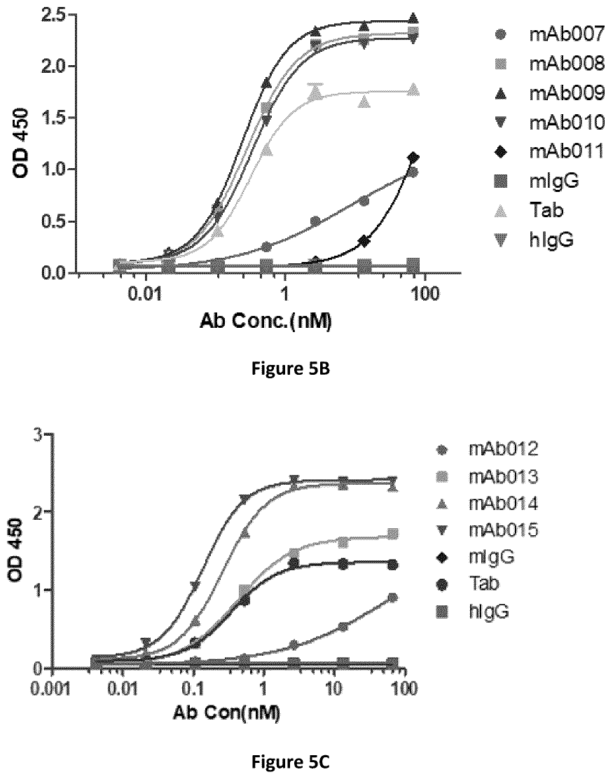

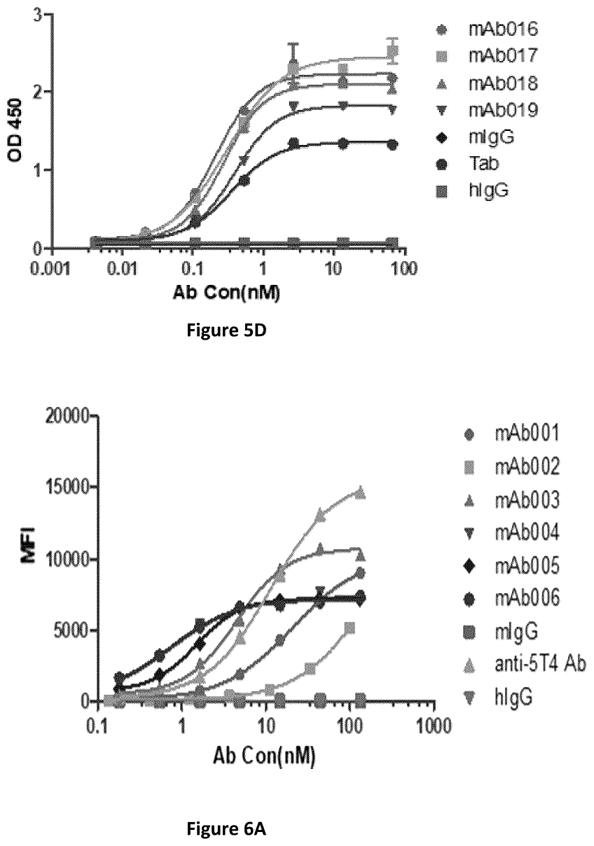

FIGS. 5A-D present the binding of the purified mAbs to the 5T4-ECD-Fc Antigen by ELISA. Excluding mAbs 2, 4, 6, 7 11 and 12, EC50 values ranged from 0.13 nM to 0.42 nM among these antibodies.

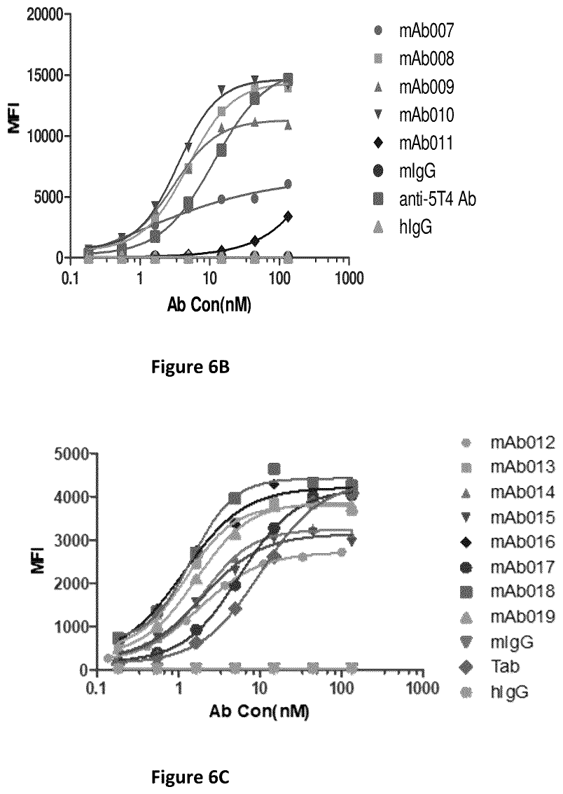

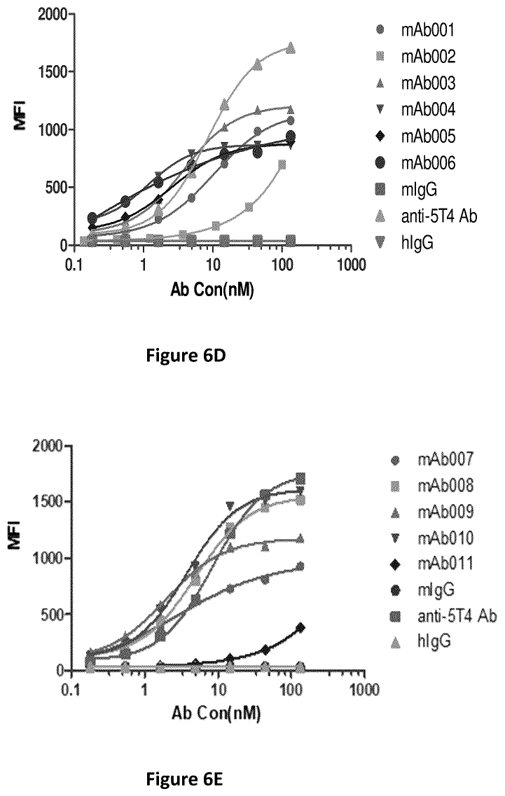

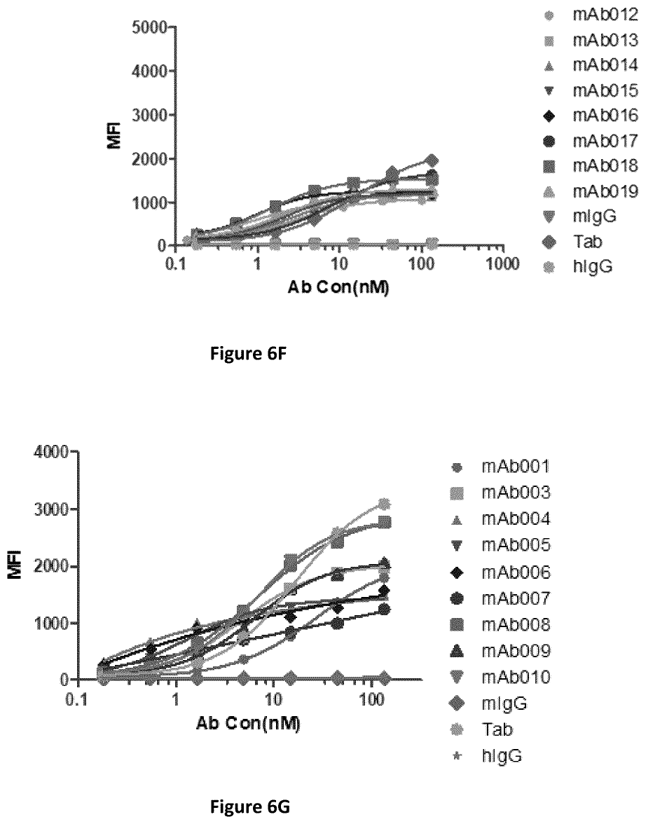

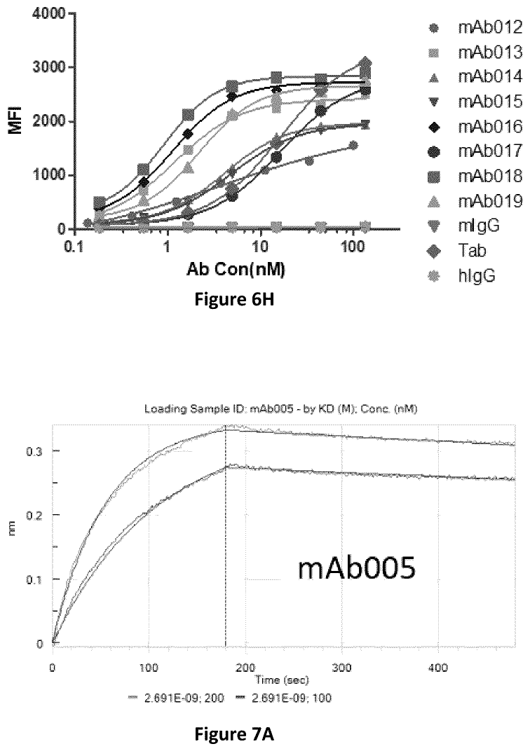

FIGS. 6A-6H present the FACS binding of selected purified mAbs to the CHO over expressing human 5T4 (FIGS. 6A-6C) and cyno 5T4 (FIGS. 6D-F) as well as to MCF-7, a breast cancer cell line endogenously express human 5T4 Antigen (FIGS. 6G-6H). As shown in FIGS. 6A-6H, excluding mAbs 001 and 017, all tested mAbs had EC50 value range of ˜ 1-4.9 nM to the CHO cells over expressing the human or the cyno 5T4, while wider range of EC50 values was observed when using MCF-7 cells endogenously express human 5T4 protein. No binding is observed on CHO parental cells (data not shown).

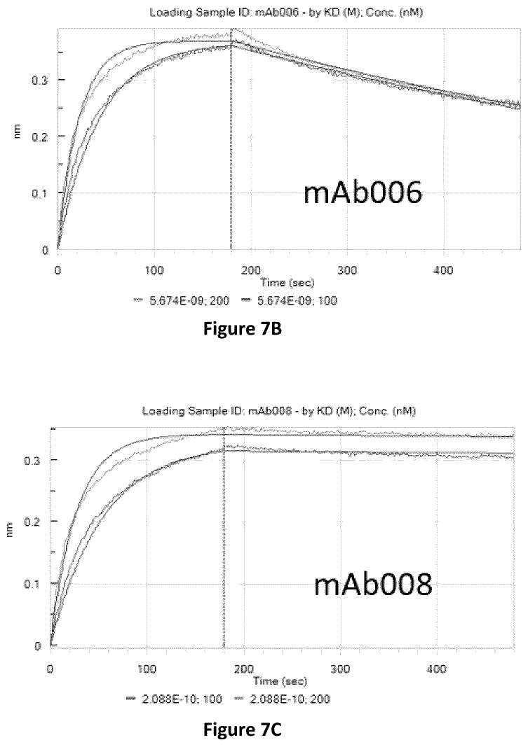

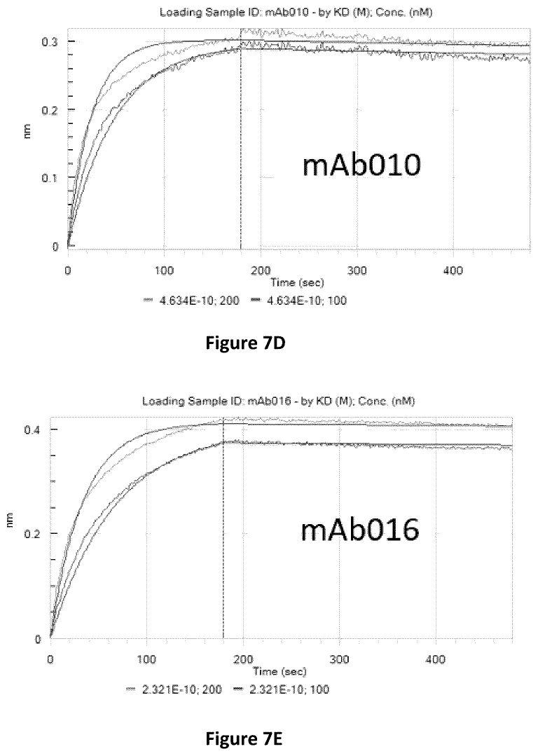

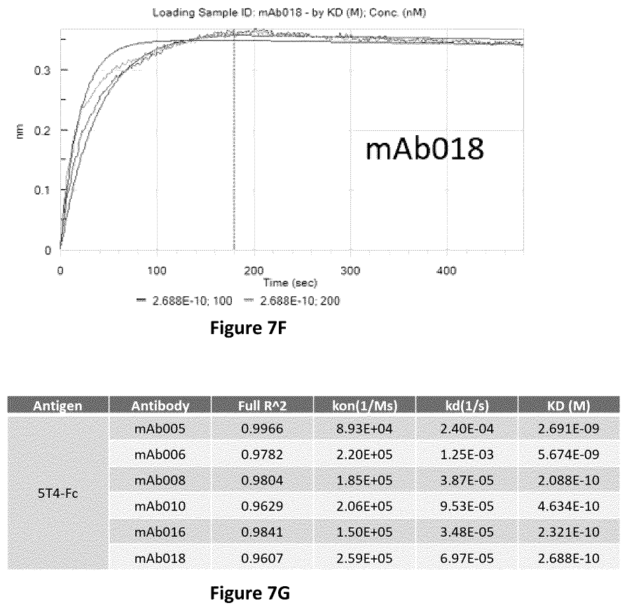

FIGS. 7A-7G present the Octet data determined the KD affinities of selected mAb. FIGS. 7A-7F demonstrate the Octet test analysis, and FIG. 7G summarize the ranked KD values of 2.088E-10M. 2.321E-10M, 2.688E-10M, 4.634E-10M, 2.691E-09M and 5.674E-09M to the following mAbs, mAb008>mAb016/mAb018>mAb010>mAb005/mAb006, respectively.

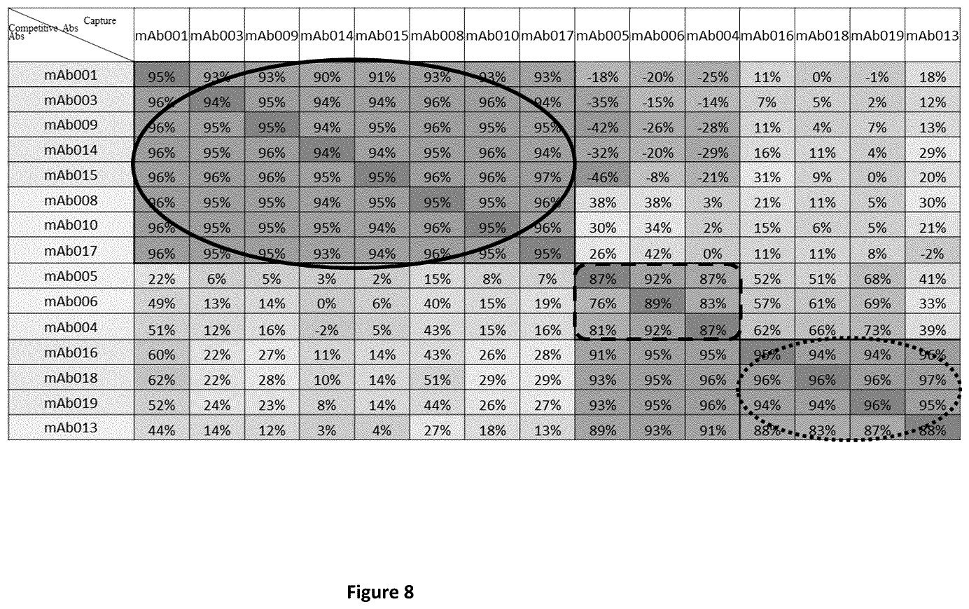

FIG. 8 summarizes the epitope binning analysis performed by ELISA. ELISA analysis suggests 3 major groups differentiated in their epitope bin as follows: Group1 (circle continuous line) gather mAb001, mAb003, mAb008, mAb009, mAb010, mAb014, mAb015 and mAb017. Group 2 (square dashed line) gather mAb004, mAb005 and mAb006, and Group 3 (circle dashed line) gather mAb013, mAb016, mAb018 and mAb019.

Summary: monoclonal antibodies (mAbs) against human 5T4 were successfully generated using hybridoma technology. 19 clones were identified and characterized. mAbs were further expressed and produced by the hybridoma clones and further purified and characterized for ELISA binding to 5T4 antigen, FACS binding to cells expressing human and cyno 5T4, affinity by Octet test and finally for epitope binning by ELISA. Two mAbs were further selected for further processing.

### Example 3

### Recombinant Production of Chimeric Mabs Anti Human 5T4 Derived from the Mouse Hybridoma Clones Against Human-5T4

Objective: Introduce the mouse CDRs sequences of the anti-human 5T4 mAbs to human IgG1 construct format or to the Tribody construct format to produce a chimeric Ab.

Methods: In brief, selected positive monoclonal hybridoma cells (˜1×10⁷) were collected for total RNA isolation following the protocol of NucleoZOL Reagent (MACHEREY-NAGEL, 740404.200). Total RNAs were used for cDNA synthesis following the kit manual of SMARTer® RACE 5′/3′, and random primer was used for the syntheses of first-strand cDNA. To amplify the heavy and light chain variable regions with PCR, synthetic cDNA was employed as template, the primers from mouse Ig-Primer Set (Novagen, 69831-3) as Gene-Specific Primer (GSP). PCR products with correct size were collected and purified with NucleoSpin® Gel and PCR Clean-up (Macherey-Nagel, 740609.250) following the Kit's manual, and subjected to TA cloning and sequencing.

The heavy chain and the light chain variable regions (VH and VL) of 5T4-mAbs were cloned into human IgG1 to make a chimeric IgG1, which were re-screened for binding by ELISA and FACS. Similarly, the VH and VL of 5T4-mAbs were cloned into Tribody and characterized by ELISA and FACS binding to 5T4.

Results: Selected hybridoma clones (mAb003, mAb006, mAb008, mAb10, mAb014, mAb016 and mAb018) were sequenced as described in the methods above and yielded variable heavy chain (HC) and variable light chain (LC) sequences for each of the clones. The variable heavy chain (HC) and the light chain (LC) sequences of mAb008 and mAb0016 were introduced either to hIgG1 expression vectors or to Tribody expression vectors, co-transfected and purified by either protein A column for the hIgG1 format, or by affinity chromatography for the Tribody format. Octet analysis confirmed affinities of 1.47E-08M for mAb0016-IgG1 and 4.52E-10M (data not shown).

Table 1 provides a reference for the data provided throughout the Examples of different embodiments of 5T4 antibodies analyzed.

TABLE 1
SEQ ID NOs, Clone Names, and Ab Component thereof.

| SEQ ID NO: | Name | Ab component |
|---|---|---|
| 1 | IM, 5T4 mouse hybridoma cl-16 | VH |
| 2 | | CDR1 |
| 3 | | CDR2 |
| 4 | | CDR3 |
| 5 | | VL |
| 6 | | CDR1 |
| 7 | | CDR2 |
| 8 | | CDR3 |
| 9 | IM, 5T4 mouse hybridoma cl-8 | VH |
| 10 | | CDR1 |
| 11 | | CDR2 |
| 12 | | CDR3 |
| 13 | | VL |
| 14 | | CDR1 |
| 15 | | CDR2 |
| 16 | | CDR3 |
| 17 | IM, 5T4 mouse hybridoma cl-3 | VH |
| 18 | | CDR1 |
| 19 | | CDR2 |
| 20 | | CDR3 |
| 21 | | VL |
| 22 | | CDR1 |
| 23 | | CDR2 |
| 24 | | CDR3 |
| 25 | IM, 5T4 mouse hybridoma cl-5 | VH |
| 26 | | CDR1 |
| 27 | | CDR2 |
| 28 | | CDR3 |
| 29 | | VL |
| 30 | | CDR1 |
| 31 | | CDR2 |
| 32 | | CDR3 |
| 33 | IM, 5T4 mouse hybridoma cl-6 | VH |
| 34 | | CDR1 |
| 35 | | CDR2 |
| 36 | | CDR3 |
| 37 | | VL |
| 38 | | CDR1 |
| 39 | | CDR2 |
| 40 | | CDR3 |
| 41 | IM, 5T4 mouse hybridoma cl-10 | VH |
| 42 | | CDR1 |
| 43 | | CDR2 |
| 44 | | CDR3 |
| 45 | | VL |
| 46 | | CDR1 |
| 47 | | CDR2 |
| 48 | | CDR3 |
| 49 | IM, 5T4 mouse hybridoma cl-14 | VH |
| 50 | | CDR1 |
| 51 | | CDR2 |
| 52 | | CDR3 |
| 53 | | VL |
| 54 | | CDR1 |
| 55 | | CDR2 |
| 56 | | CDR3 |
| 57 | IM, 5T4 mouse hybridoma cl-18 | VH |
| 58 | | CDR1 |
| 59 | | CDR2 |
| 60 | | CDR3 |
| 61 | | VL |
| 62 | | CDR1 |
| 63 | | CDR2 |
| 64 | | CDR3 |

IM, 5T4 humanized sequences (based on cl-8 and cl-16 CDRs)

| | | |
|---|---|---|
| 65 | 5T4_IM_11 | VH |
| 66 | | VL |
| 67 | 5T4_IM_12 | VH |
| 68 | | VL |
| 69 | 5T4_IM_13 | VH |
| 70 | | VL |
| 71 | 5T4_IM_14 | VH |
| 72 | | VL |
| 73 | 5T4_IM_15 | VH |
| 74 | | VL |
| 75 | 5T4_IM_16 | VH |
| 76 | | VL |
| 77 | 5T4_IM_17 | VH |
| 78 | | VL |
| 79 | 5T4_IM_18 | VH |
| 80 | | VL |
| 81 | 5T4_IM_19 | VH |
| 82 | | VL |
| 83 | 5T4_IM_20 | VH |
| 84 | | VL |
| 85 | 5T4_IM_21; 5T4_IM_22 | VH |
| 86 | | VL |
| 87 | 5T4_IM_23; 5T4_IM_24 | VH |
| 88 | | VL |
| 89 | 5T4_IM_25; 5T4_IM_26 | VH |
| 90 | | VL |
| 91 | 5T4_IM_27; 5T4_IM_28 | VH |
| 92 | | VL |
| 93 | 5T4_IM_29 | VH |
| 94 | | VL |
| 95 | 5T4_IM_30 | VH |
| 96 | | VL |
| 97 | 5T4_IM_31 | VH |
| 98 | | VL |
| 99 | 5T4_IM_32 | VH |
| 100 | | VL |
| 101 | 5T4_IM_33 | VH |
| 102 | | VL |
| 103 | 5T4_IM_34 | VH |
| 104 | | VL |
| 105 | 5T4_IM_35 | VH |
| 106 | | VL |
| 107 | 5T4_IM_36 | VH |
| 108 | | VL |
| 109 | 5T4_IM_37 | VH |
| 110 | | VL |
| 111 | 5T4_IM_38 | VH |
| 112 | | VL |
| 113 | 5T4_IM_39 | VH |
| 114 | | VL |
| 115 | 5T4_IM_40 | VH |
| 116 | | VL |
| 117 | 5T4_IM_41 | VH |
| 118 | | VL |
| 119 | 5T4_IM_42 | VH |
| 120 | | VL |
| 121 | 5T4_IM_43 | VH |
| 122 | | VL |
| 123 | 5T4_IM_44 | VH |
| 124 | | VL |
| 125 | 5T4_IM_45 | VH |
| 126 | | VL |
| 127 | 5T4_IM_46 | VH |
| 128 | | VL |
| 129 | 5T4_IM_47 | VH |
| 130 | | VL |
| 131 | 5T4_IM_48 | VH |
| 132 | | VL |
| 133 | 5T4_IM_49 | VH |
| 134 | | VL |
| 135 | 5T4_IM_50 | VH |
| 136 | | VL |
| 137 | 5T4_IM_51 | VH |
| 138 | | VL |
| 139 | 5T4_IM_52 | VH |
| 140 | | VL |
| 141 | 5T4_IM_53 | VH |
| 142 | | VL |
| 143 | 5T4_IM_54 | VH |
| 144 | | VL |
| 145 | 5T4_IM_55 | VH |
| 146 | | VL |
| 147 | 5T4_IM_56 | VH |
| 148 | | VL |
| 149 | 5T4_IM_57 | VH |
| 150 | | VL |
| 151 | 5T4_IM_58 | VH |
| 152 | | VL |
| 153 | 5T4 ScFv | VH |
| 154 | | CDR1 |
| 155 | | CDR2 |
| 156 | | CDR3 |
| 157 | | VL |
| 158 | | CDR1 |
| 159 | | CDR2 |
| 160 | | CDR3 |

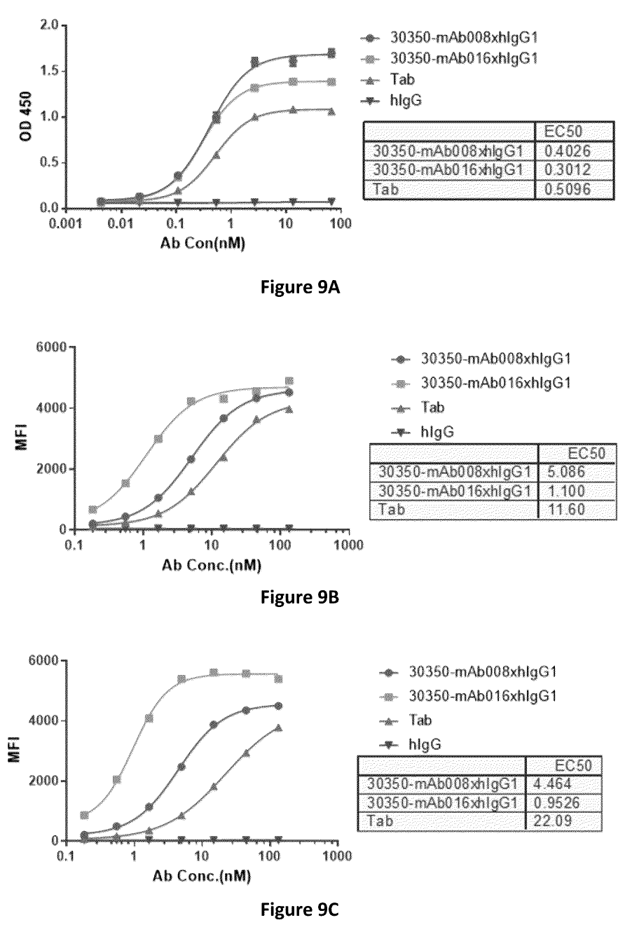

The chimeric hIgG1Abs were tested for ELISA and FACS binding. EC50 of 0.4 nM and 0.3 nM for mAb008-hIgG1 and mAb016-hIgG1 chimeric Abs, respectively, were observed when binding was performed on recombinant 5T4 protein by ELISA (FIG. 9A). EC50 of 5 nM and 1.1 nM for mAb008-hIgG1 and mAb016-hIgG1 chimeric Abs, respectively, were observed when binding was performed on CHO over expressing human 5T4 protein by FACS (FIG. 9B). Similar binding data was observed when FACS binding was performed on MCF-7 cell line endogenously expressing 5T4 protein, with EC50 of 4.4 nM and 0.9 nM for mAb008-hIgG1 and mAb016-hIgG1 chimeric Abs, respectively (FIG. 9C). Similar FACS binding data was observed when binding was performed on CHO over expressing cyno 5T4 protein with EC50 of 5.7 nM and 2.2 nM for mAb008-hIgG1 and mAb016-hIgG1 chimeric Abs, respectively (data not shown).

Similarly, ELISA and FACS binding were also validated for the Chimeric Tribody formats (data not shown).

Summary: The mouse Light chain variable domains (VL) and Heavy chain variable domains (VH) were sequenced from the selected hybridoma clones and were converted into scFvs and synthesized as components of either hIgG or Tribody constructs to form chimeric molecules. These molecules were expressed, purified, and were further characterized by binding assays and showed that the binding characteristics to human5T4 was maintained.

### Example 4

### Humanization, Expression, Purification and Characterization of Fully Humanized Tribody Antibody Constructs

Objective: To express purify and select a fully humanized Tribody trispecific antibody construct following humanization of mouse anti human5T4 sequences.

Methods: Humanization: Briefly, the CDR residues within the heavy chain and the light chain variable region (VH/VL CDRs) of mAb016, were determined and annotated by Kabat numbering system. After analysis, the risky hot spot of deamidation motif (NG) was identified in CDR H2 region, which was designed to mutate to QG, SG, NA.

Two separate Ig BLAST searches were performed for VH and VL of mAb016. The human germlines IGHV-2*02 and IGKV3-11*01 with high identity of framework sequence to mAb016 were chosen as the acceptor human germline framework for grafting VH and VL CDRs, respectively. Meanwhile, JH6 and JK4 were selected as the J-region of VH and VL based on best sequence homology.

BioLuminate modeling package of Schrodinger suite software was used for building homology model. Antibody structure 2W9D (PDB code) was selected as template for heavy and light-chain modeling. The model generated by BioLuminate was further analyzed in order to identify the residues in framework regions that potentially support CDR loop structures and VH/VL interface. Those residues that could have an impact on CDR loop conformation and VH/VL interface were backmutated. Finally, a panel of humanized variants for mAb016 were designed and were constructed in the Tribody format.

An additional humanization strategy was performed. Briefly, mabHuman technology was used to graft the CDRs of mAb008 and mAb016 into new antibodies' frameworks. Various VH and VL sequences have been found within the human antibody framework sequence database, based on NGS experiments following sequences alignment.

Gene Synthesis And Plasmid Construction. The coding sequences for the heavy chain (HC) and light chain (LC) of the trispecific antibody were generated by DNA synthesis and PCR, subsequently subcloned into pCDNA3.4-based plasmid (Invitrogen) for protein expression in mammalian cell system. Finally, the gene sequences in the expression vectors were confirmed by DNA sequencing.

Expression of Trispecific Antibody Construct. Transient expression of the Tribody/Pro-Tribody antibodies was performed by co-transfection of paired HC and LC constructs (at 1:1 HC/LC ratio for the Tribody format or 2.5:1 HC/LC ratio for the ProTribody format) into CHO cells using PEI method. Briefly, 1 L of CHO cells at approximately 5.5×10⁶/ml in a 3 L shake flask was used as the host, Transfection was initiated by adding a mixture of 1 mg of total DNA and 4 mg PEI in 100 ml OptiMEM medium (Invitrogen) to the cells and gentle mixing. Cells were then cultured in an incubator shaker at 120 rpm, 37° C., and 8% CO₂, for 8-10 days. Feeding with peptone and glucose was carried out 24 h later and every 2-3 days thereafter depending on the cell density and viability. The cell culture was terminated on day 8-10 when cell viability reduced to <80%. The conditioned medium was harvested for protein purification.

Purification of Trispecific Antibody Construct. Protein purification by affinity chromatography and SEC was performed using an AKTA pure instrument (GE Lifesciences). Affinity capture of the Tribody was achieved by passing the harvested supernatants over a column of CaptureSelect™ CH1-XL Affinity Matrix (Thermo Scientific). After washing column with Buffer A (25 mM Tris, 150 mM NaCl, 5 mM EDTA, pH 7.5), the protein was eluted with Buffer B (50 mM Sodium citrate, 150 mM NaCl, pH 3.0), and immediately neutralized with ⅙ volume of Buffer D (1 M Aarginine, 400 mM Succinic acid, pH 9.0). The affinity purified protein was then concentrated to 5-10 mg/ml using Amicon 30 kD concentrator (Merck Millipore) and subjected to SEC purification on a Superdex200 column (GE Lifesciences) equilibrated with SEC Buffer: 200 mM Arginine, 137 mM Succinic acid, 0.05% Tween-80,150 mM NaCl, pH5.0. The target tribody fractions were collected, then added 5% trehalose (146 mM). The Tribody product was analyzed using SDS-PAGE and HPLC-SEC.

SDS-PAGE Analysis of Trispecific Antibody Construct. SDS-PAGE analysis of Tribody was carried out under reducing and non-reducing conditions in pre-cast polyacrylamide gels. Briefly, 2 μg Tribody samples were mixed by NuPAGE™ LDS sample buffer (thermofisher-NP0008) with 70 mM DTT add or not. After incubating at 25° C. or 90° C. for 10 min, the samples and Unstained Protein Standards (BIO RAD-161-0363 were loaded onto the gels. Electrophoresis was carried out at a constant voltage of 120 V with 1×Tris-glycine-SDS running buffer. Following electrophoresis, gels were stained for overnight using Coomassie blue and de-stained with destaining solution (10% acetic acid, 40% methanol and 50% water). Destained gels were scanned with a Gel imaging system (Tanon-2500R).

SEC-HPLC Analysis of Precursor Trispecific Antibody Construct. Analytical SEC-HPLC was performed using Shimadzu LC-10 HPLC instrument (Shimadzu Corp.). 20 μl sample on 1 mg/ml will be loaded to a Superdex 200 Increase 5/150GL column (GE Lifesciences). The mobile phase was 2*PBS with a flow rate of 0.3 ml/min, 15 min.

LC-MS Analysis of Tribody Construct. The Tribody was separated with ACQUITY UPLC BEH200 Å, SEC column (Waters 1.7 μm, 4.6×300 mm) at room temperature and detected by ESI-MS (Thermo, MS-B20-03). The mobile phase was 0.1% formic acid: acetonitrile (75:25, v/v) with a flow rate of 0.2 mL/min. Mass spectrometry was performed in the positive ion. Other parameters for mass spectrometry were: resolution of 17500, Scan range of 1000-5000 m/z, In-source CID of 60 eV, sheath gas flow rate of 30 L/min, capillary temperature of 350° C., Spray voltage of 2.5 KV.

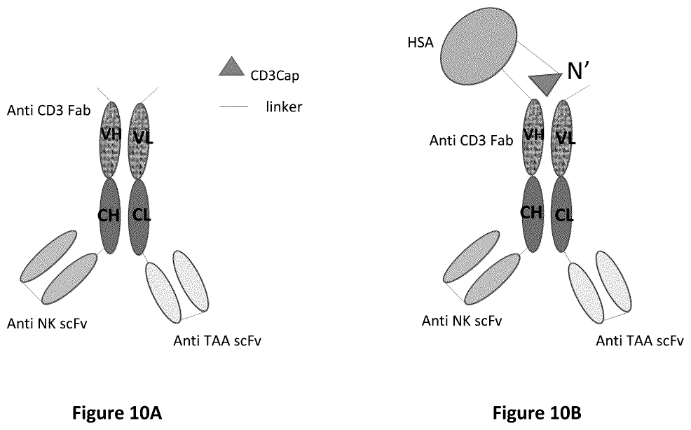

Results: The humanization process of 5T4 mAbs from both strategies yielded new Tribody sequences as follows: IM-1100-1109 molecules, corresponding to 5T4_IM11-20 sequences, IM-1198-1227 molecules, corresponding to 5T4_IM29-58 sequences, and IM-1175-1182 molecules corresponding to 5T4_IM21-28 sequences, where the HC of the Tribody molecule is comprised of anti CD3 heavy chain (VH-CH) fused to anti NKengager (scFv), and the LC of the Tribody molecule is comprised of CD3 light chain (VL-CL) fused to anti 5T4 (VH-VL/VL-VH) as schematically shown in FIG. 10A. (For the 5T4 clone names and related SEQ ID NOs: see Table 1 above). In FIG. 10B a ProTribody structure is demonstrated with additional regulatory domains such as CAP masking domain, HSA moiety and a protease cleavage linker to generate a conditionally activated molecule previously described for example see WO 2020/225805, incorporated herein in full.

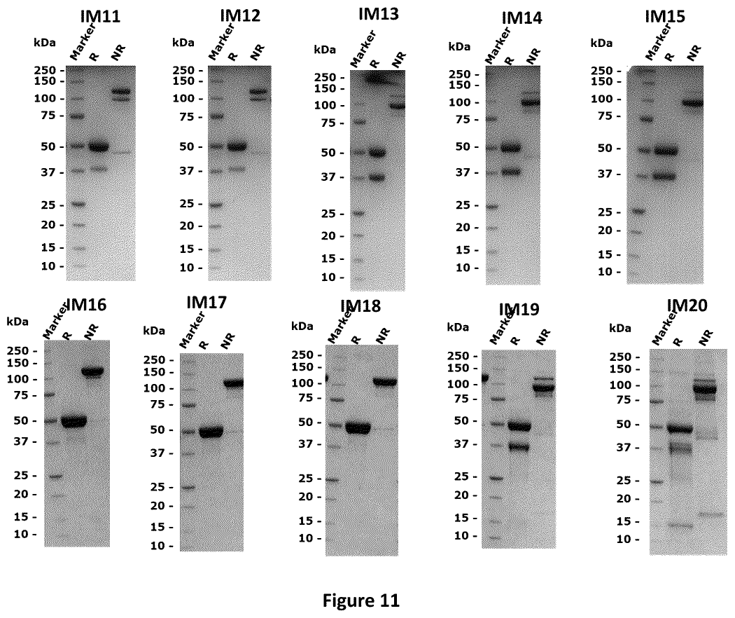

FIG. 11 show variety of SDS-PAGE analysis of various constructs to demonstrate wide range of quality of the expressed proteins.

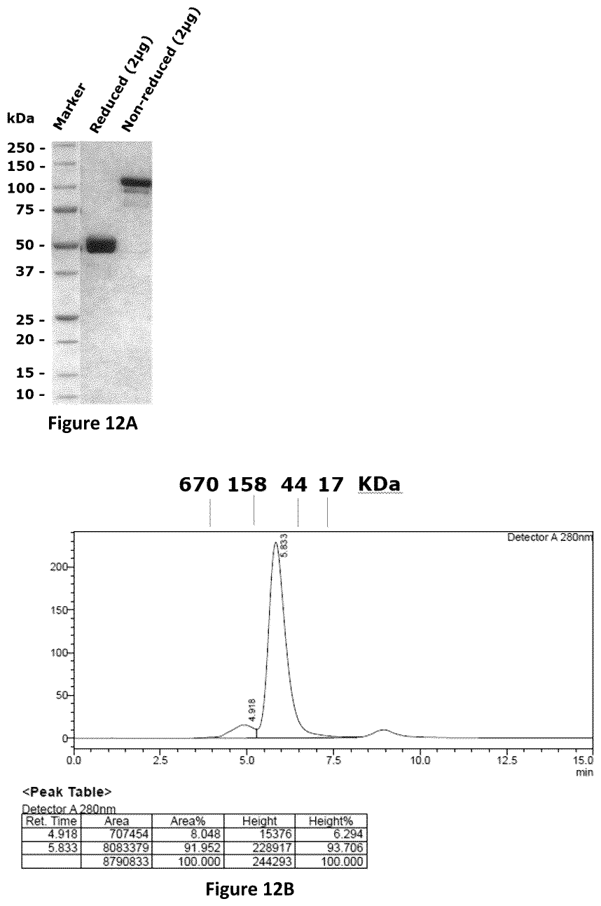

The expressed HC and LC Tribody IM-1222 constructs were associate to form a single molecule, as indicated by the single ˜100 kDA band observed in the SDS-PAGE (in non-reduced conditions), and by a single major peak at retention time of ˜5.8 min in SEC-HPLC (FIGS. 12A and 12B, respectively). These results are in agreement with the expected retention time of the expected MW based on mass calibration curve.

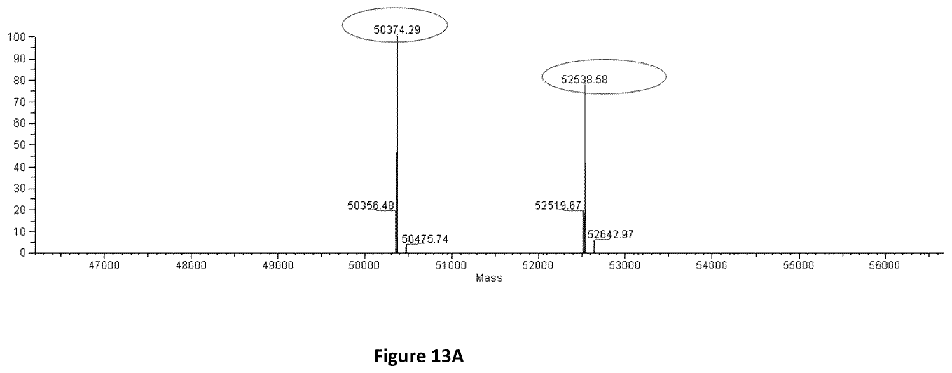

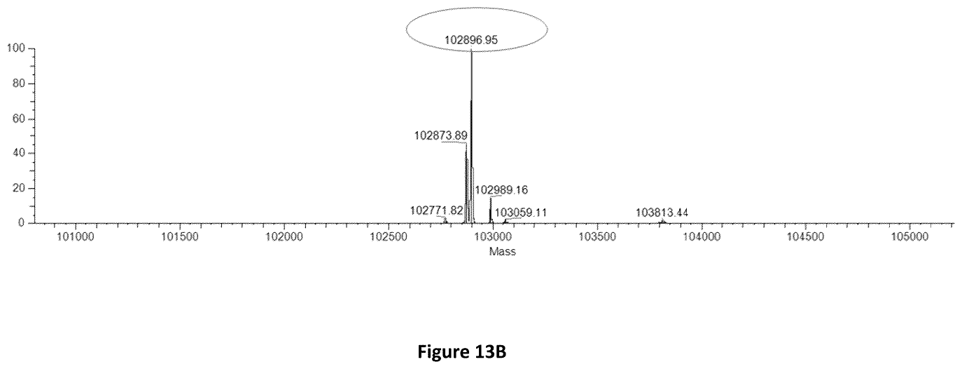

MS analysis of the Tribody IM-1222 constructs confirmed a 50 kDa peak for the LC, a 52 kD peak for the HC in reduced conditions (FIG. 13A) and 102 kDa for the intact protein (FIG. 13B).

The expressed HC and LC Tribody IM-1178 constructs were associate to form a single molecule, as indicated by the single ˜100 kDA band observed in the SDS-PAGE (in non-reduced conditions), and by a major peak at retention time of ˜5.8 min in SEC-HPLC (FIGS. 14A and 14B, respectively). These results are in agreement with the expected retention time of the expected Mw based on mass calibration curve.

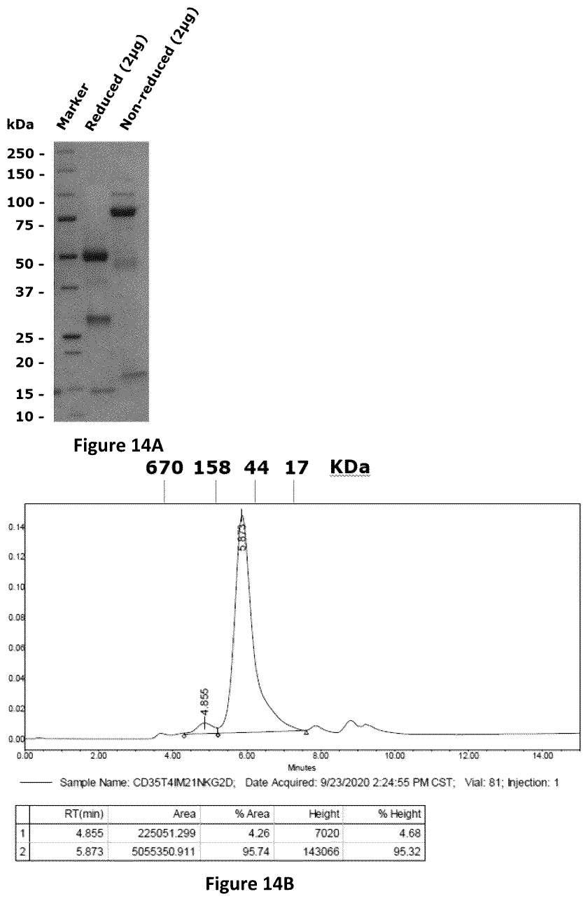

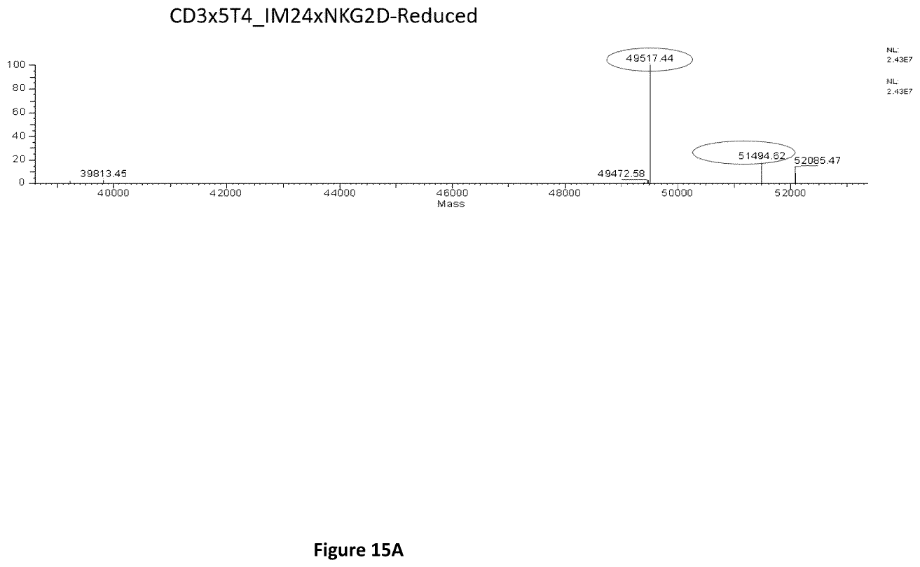

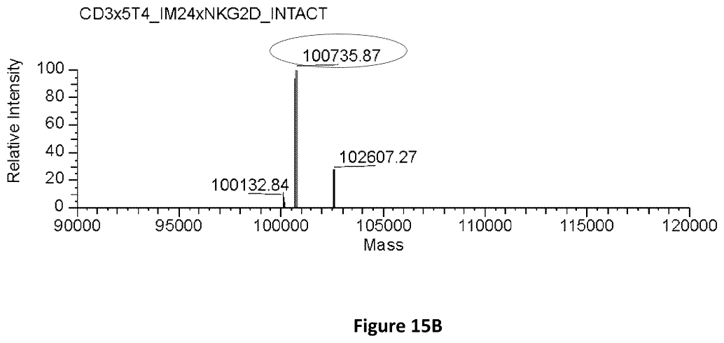

MS analysis of the Tribody IM-1178 constructs confirmed a 49 kDa pick for the LC, a 51 kD pick for the HC in reduced conditions (FIG. 15A) and 100 kDa for the intact protein (FIG. 15B).

Summary: Multiple molecules of the 5T4 humanized sequences in the Tribody structure were expressed purified and analyzed by SDS-PAGE, SEC-HPLC and Mass-Spec. The various products had various qualities in terms of purity and yield. FIGS. 13A-13B demonstrate an example of selected construct, IM-1222 Tribody which correspond to 5T4_IM53 sequence derived from mAb016. FIGS. 14A-14B demonstrate an example of selected construct, IM-1178 Tribody which correspond to 5T4_IM24 sequence derived from mAb008 which include CD3x5T4_IM24xNKG2D.

### Example 5

### Binding of Fully Humanized Tribody Antibody Constructs to Recombinant Proteins by ELISA

Objective: To study the binding efficacy of the humanized Tribody antibody constructs to 5T4 by ELISA. These constructs comprised of the anti TAA ScFv domain (anti 5T4 humanized), T cell engager domain (anti CD38 Fab), and NK engager domain (anti NKG2A/anti NKG2D). Additional variants may be comprised of a CAP masking sequences, a cleavable linker, a non-cleavable linker, as well as a point-mutated engager sequences that are lack of binding activity to the specific engager and serve as negative controls for the Tribody/Protribody variants.

Methods: ELISA Binding of Tribody Antibody Constructs To Antigens: Dilute target protein h5T4-His (cat #19845-H08H, supplier Sino Biological) into PBS with final concentration of 0.3 μg/mL and coat 100 L/well on ELISA plate (cat: 9018, supplier Corning). Incubate O/N, 4° C. The plates were blocked with 250 μL 1% BSA in PBST for 1 hr at 37° C. Wash 4 times with PBST. All the washes are done using Biotek (Elx 405). All the Tribody set antibodies were diluted to 400 nM and make 4-fold serially dilutions (12 points, including 0 point). Add 100 μL/well diluted antibody constructs solution to plate, incubate for 1 hr at 37° C. Wash 4 times with PBST. Add 100 μL/well anti-human kappa light chain-HRP (1:10000), incubate for 0.5 hr at 37° C. Wash 4 times with PBST. 100 μL/well of TMB substrate was added and incubated at room temperature for 5 min. 100 μL/well of 1N HCl to terminate reaction. Plates were read using ELISA plate reader at 450 nm wavelength (instrument SpectraMax M5e). Data Analysis was performed using Graphpad prism 5 software by using nonlinear regression (curve fit): log (agonist) vs. response, agonist is antibody concentration (nM) and response is OD value.

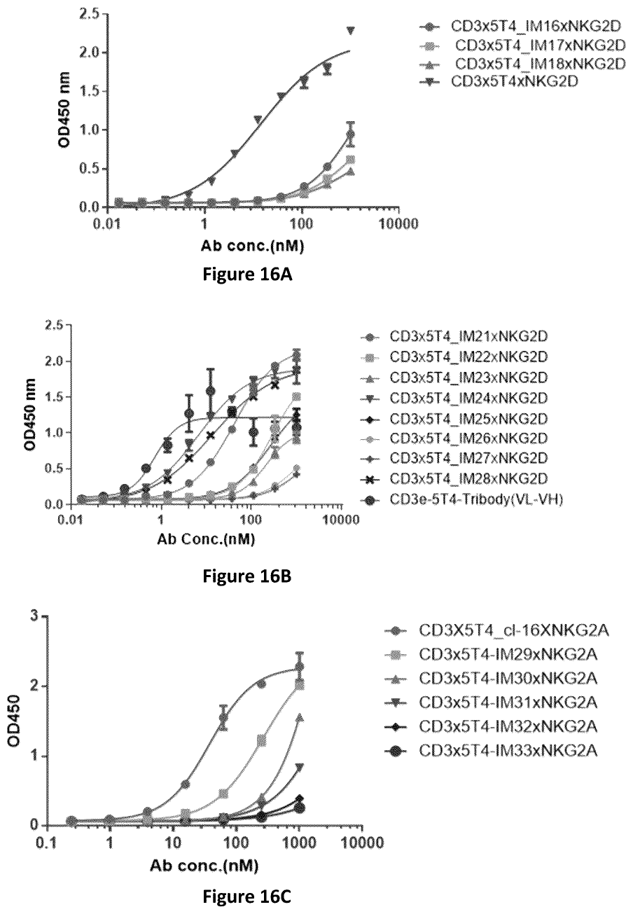

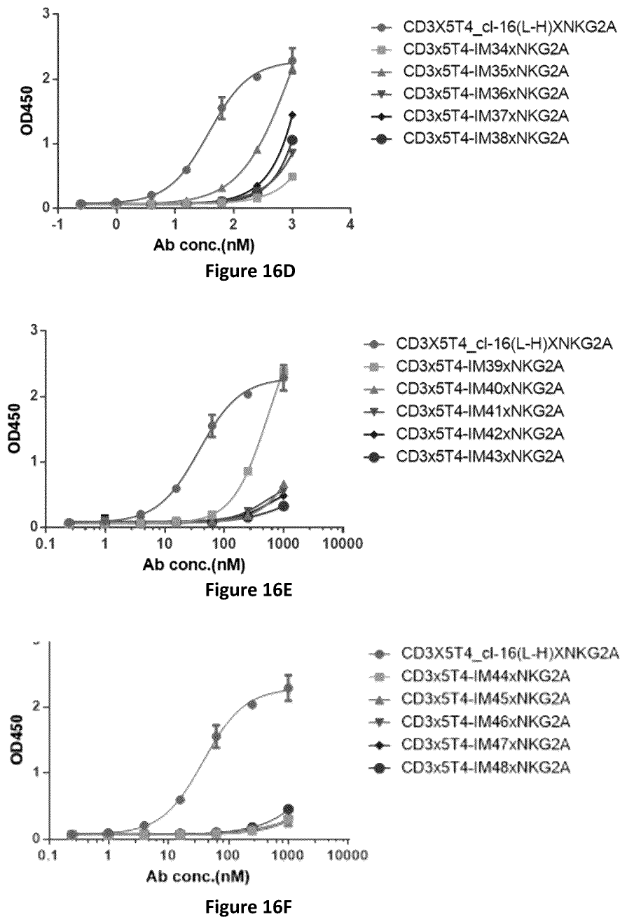

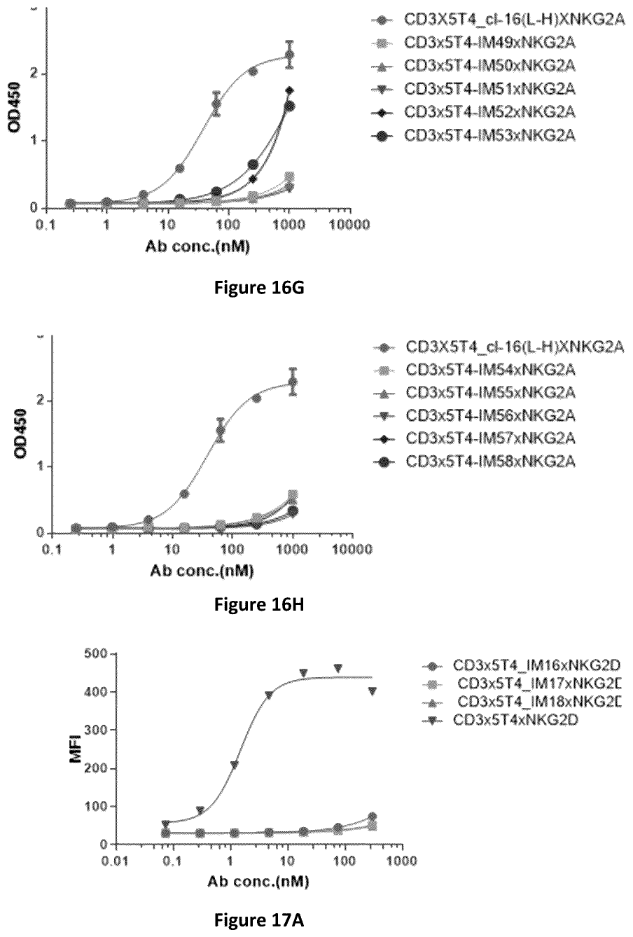

Results: The expressed trispecific constructs that are comprised of humanized 5T4 sequences were analyzed for their binding efficacy to 5T4 protein. A wide range of EC50 values of the humanized 5T4 sequences (5T4_IM11-20 or 5T4_IM29-58) to human 5T4 protein were observed (FIG. 16). Most variants exhibit lower EC50 values than the original non-humanized clone.

Conclusion: As shown in FIGS. 16A-16H, the binding of the Tribody that harbors the humanized 5T4 sequences was confirmed in some of the humanized constructs, while other constructs had low EC50 values, and some constructs lacked binding to 5T4 recombinant antigen protein. The fact that different frameworks result with different binding affinities towards h5T4 may be a result of compatibility of the CDRs to a specific framework, which may result in structural perturbations that hamper correct CDR orientation.

### Example 6

### Binding of Fully Humanized Trispecific Antibody Constructs to Cells by FACS

Objective: To study the binding efficacy of the humanized Tribody antibody constructs to cells expressing membrane bound endogenous 5T4 (NCI-H226) or ectopic 5T4 (CHO cells over expressing 5T4), by FACS. These constructs comprised of the anti TAA ScFv domain (anti 5T4 humanized), Tcell engager domain (anti CD38 Fab), and NK engager domain (anti NKG2A/anti NKG2D). Additional variants may be comprised of a CAP masking sequences, a cleavable linker, a non-cleavable linker, as well as a point-mutated engager sequences that are lack of binding activity to the specific engager and serve as negative controls for the Tribody/Protribody variants.

Methods: FACS Binding of Tribody Antibody Constructs to Cells. Suspension cultured cells was harvested directly, and adherent cell were digested using TrypLE Express Enzyme (cat: 12604-013, supplier Life technologies). Centrifuge at 1000 rpm for 5 min and discard the supernatant. Cells are suspended at a concentration of 2×10⁶/mL in FACS buffer (2% FBS in PBS) and add 100 μL/well of cell suspension to the plate (cat #3799, supplier Corning). Centrifuging the plates at 2000 rpm for 5 min and discard the supernatant. Re-suspend the cells in 100 μL/well of Tribody set antibodies (400 nM start, 4-fold dilution, 8 point including 0 point) and incubate the plate for 60 min at 4° C. Centrifuge the plate at 2000 rpm, 4° C. for 5 min and discard supernatant. Then wash the cells 3 times with 170 μL FACS buffer. Re-suspend the cells at 100 μL/well with secondary antibody (goat anti-human Ig Fab-FITC, Cat #2085-02, Southern biotech) with 1:400 dilution and incubate the plate for 30 min at 4° C. in dark. Centrifuge the plate at 2000 rpm, 4° C. for 5 min and discard supernatant. Then wash the cells 3 times with FACS buffer and analyze the sample with FACS verse. The fluorescence intensity of the staining was measured using flow cytometer (BD, FACSVerse). The geometric mean fluorescence intensity (GMFI; median fluorescence intensity (MFI)) of set antibodies staining was calculated (BD FACSuite software). Dose-response curves were generated and EC50s for the trispecific variants binding were calculated using GraphPad Prism software.

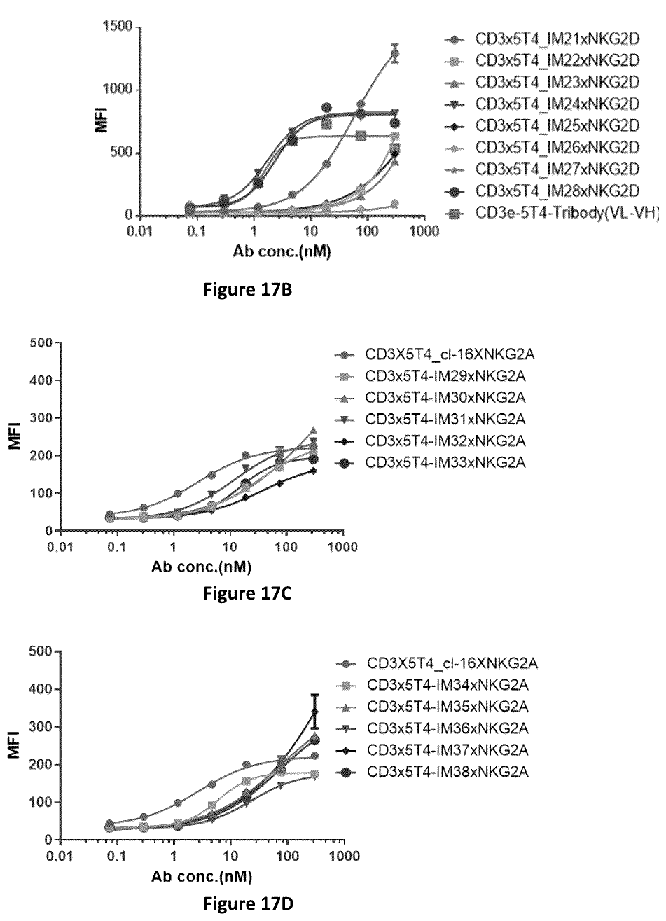

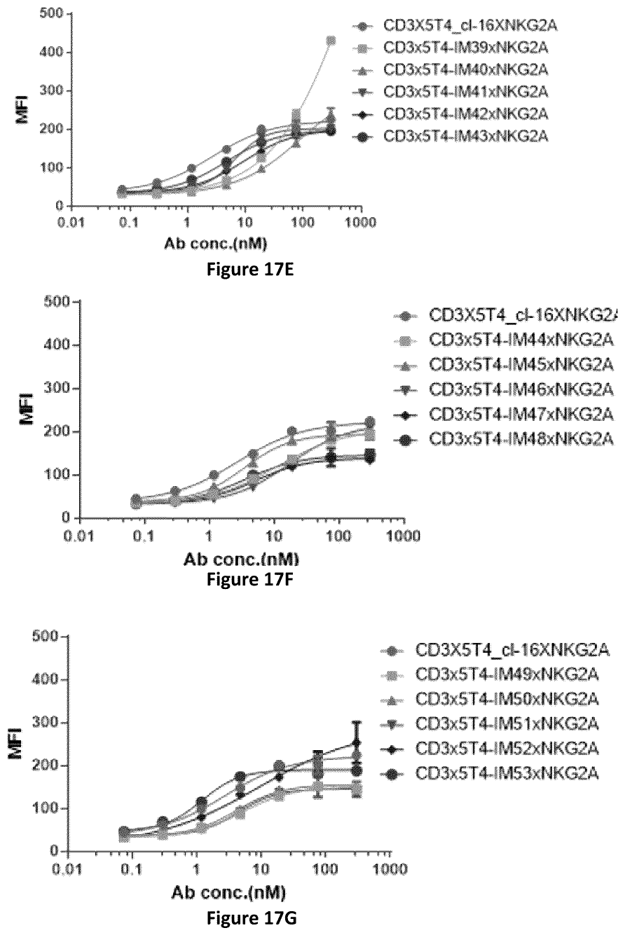

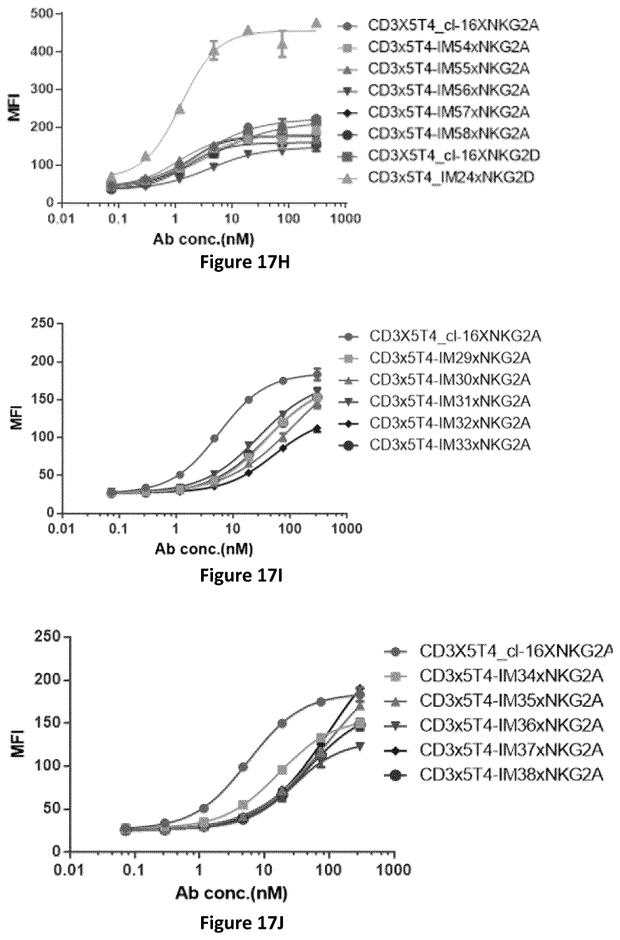

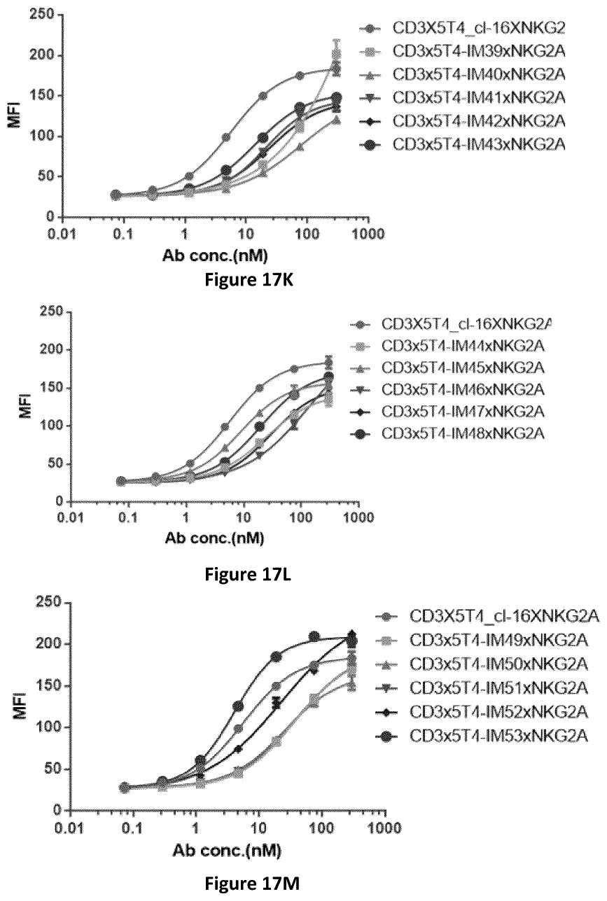

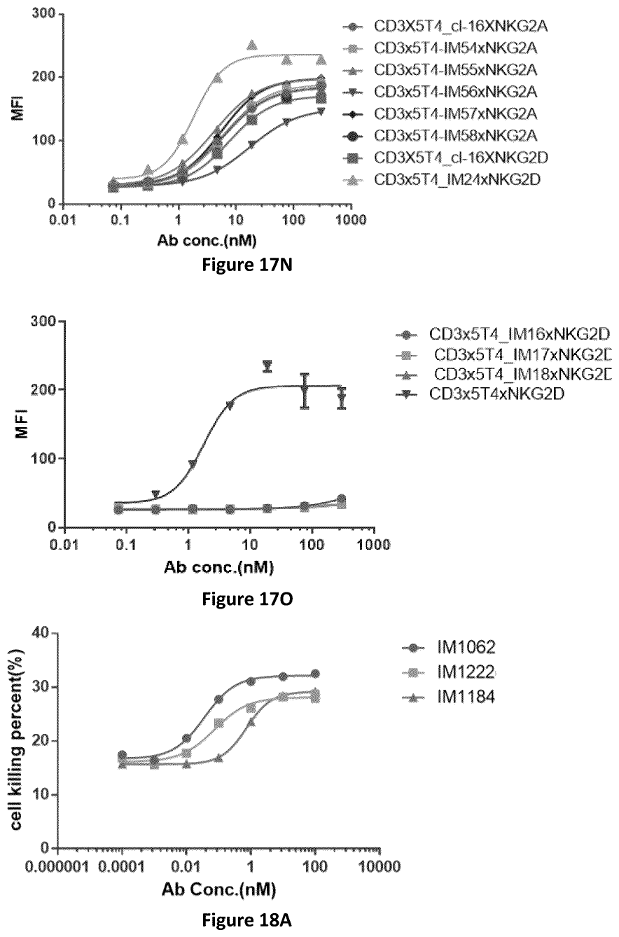

Results: The expressed trispecific constructs that are comprised of humanized 5T4 were analyzed and confirmed for their binding efficacy to CHO cells over expressing 5T4 (FIGS. 17A-17H) while no binding was observed on CHO parental cells (data not shown). Binding was also confirmed on NCI-H226 cells expressing endogenous 5T4 (FIGS. 17I-17O). The data indicates for a wide range of EC50 values observed in the various variants. Specifically, EC50 of 1.4 nM for IM-1178 and 2.3 nM for IM-1222 Tribody variants were determined on CHO cells over expressing 5T4, and EC50 of 1.24 nM for IM-1178 and 3.9 nM for IM-1222 Tribody variants were determined on NCI-H226 cells. Selected constructs were also evaluated for their binding to cells over expressing NKG2A and confirmed that the 5T4 humanized sequences did not affect the binding to NKG2A (data not shown).

Conclusion: As shown in FIGS. 17A-17O, the binding of the Tribody variants that harbors the humanized 5T4 sequences to cells expressing 5T4 was confirmed in some of the humanized constructs, while other constructs had low EC50 values, and some constructs were lack of binding. Selected construct Tribody IM-1222 correspond to 5T4_IM53 sequence was further characterized in vitro and in vivo efficacy.

### Example 7

### In Vitro Functional Evaluation of Fully Humanized Tribody Antibody Construct

Objective: To evaluate in vitro, dose dependent T-cell mediated cytotoxicity of Tribody/ProTribody variants on breast and lung cancer cells (MDA-MB-231 and NCI-H226, respectively).

Methods: Lactate Dehydrogenase (LDH) Cytotoxicity Assay. Tribody and ProTribody variants were analyzed for their potential to induce Tcells cell-mediated cytotoxicity in 5T4 expressing cancer cells. Briefly, Isolate T cell using EasySep Human T Cell Isolation Kit (STEMCELL, Cat: 17951). Adjust concentration of target cell to 2×10⁵/mL in assay buffer (blank RPMI 1640, Gibco, Cat-10491 plus 5% FBS), and add 50 μL to wells of a round-bottom 96-well plate (Cat-3799, Corning). Adjust concentration of effect cell (Isolated T cells or PBMCs from ALLCELLS) to 2E6/mL in assay buffer, and add 50 μL to wells with ET ratio of 10:1. Then add 100 L/well of 2-fold diluted antibodies, mix sufficiently. Incubate at 37° C., 5% CO₂ for 24 hr. Centrifuge the plate at 300 g for 5 min and collect supernatant. LDH release would be tested by CytoTox 96 Non-radioactive cytotoxicity assay kit (Promega, G1780). Add 20 μL lysis solution (10*) to max well, mix sufficiently and incubate at 37° C. for 45 min. Then transfer 50 μL aliquots from all test and control wells to a fresh 96-well flat clear bottom plate (Cat-3599, Corning). Add 50 μL CytoTox reagent to each well. Protect plates from light and incubate for 30 min at room temperature. Finally add 50 μL Stop Solution to each well of the 96-well plate. Record the absorbance at 490 nm or 492 nm within 1 hr after adding the Stop Solution. The result of

$$Calculation is \% Cytotoxicity = ( Experimental - E only - T only ) / ( T Max - T only ) \times 100 ) .$$

Results: Using lactate dehydrogenase assay, T cells mediated cytotoxicity was achieved for NCI-H226 (FIG. 18A) or MDA-MB-231 (FIG. 18B), respectively, with EC50 of 0.02 nM for IM-1222 Tribody (squares) and 0.38 nM for IM-1062 Tribody (circles).

Conclusion: In T cells mediated cell cytotoxicity assays, two cancer cell lines were undergoing cell killing in the presence of Tribody IM-1062 and IM222, with higher efficacy for IM-1222 represent 5T4_IM-53 humanized sequence derived from mAb 016 previously described.

### Example 8

### In Vivo Efficacy in Xenograft NSG Mouse Model

Objective: To study efficacy of IM-1222 Tribody 5T4_IM53-CD3-NKG2A on hPBMC engrafted NCI-H226 model in NCG mice and examine the inhibition of tumor growth induced by the IM-1222 Tribody in humanized mouse model.

Methods: In-vivo xenograft assay. The NCI-H226 (human lung cancer, ATCC, Cat No. CRL-5826, Lot No. 58094746) cells maintained in vitro as a monolayer culture in RPMI 1640 supplemented with 10% FBS, 100 U/ml penicillin and 100 μg/ml streptomycin maintained at 37° C. in an atmosphere containing 5% CO₂ in air. The cells growing in an exponential growth phase will be harvested and counted for tumor inoculation. NCG mice female, 6-7 weeks, weighing approximately 19-21 g were purchased from GemPharmatech Co., LTD. Mice were inoculated subcutaneously at the right flank with 5×10\hat{ }6 NCI-H226 cells in 0.2 ml mixture (base medium: Matrigel=100 ul: 100 ul) for tumor development) Day0). On Day 7 mice were i.v. injected with 1×10\hat{ }7 hPBMC from two healthy donors. For each arm half of the animals were PBMCs injected from one donor and half of the animals were injected with the second donor. The treatments started when tumors reach average size ˜150 mm3 at around one week post cell inoculation when matrigel is fully absorbed for tumor efficacy study. Mice were daily dosed (I.p) with 20 μg/Kg. Tumor sizes were measured twice weekly in two dimensions using a caliper, and the volume was expressed in mm3 using the formula: V=0.5 a×b² where a and b are the longest and shortest diameters of the tumor, respectively. The tumor sizes are then used for the calculations of tumor growth inhibition (TGI). Specifically, the study included 10 animals that were used as a vehicle arm where only TT2 buffer (200 mM Arginine, 137 mM Succinic acid, 5% trehalose, 0.05% Tween-80, pH5.0, 150 mM NaCl) was injected, and two additional arms of 6 mice each, where IM-1062 or IM-1222 were injected.

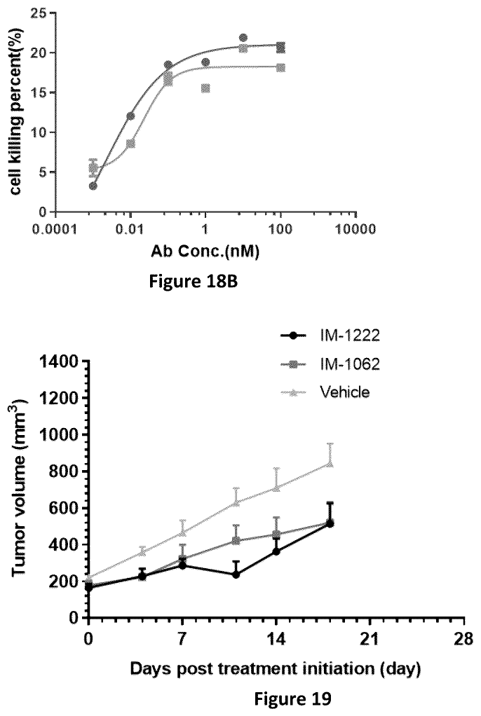

Results: FIG. 19 presents the tumor volume (mm3) for the mice treated with Tribody IM-1222 (circles), mice treated with Tribody IM-1062 (squares) and TT2 buffer control (triangles). As shown in FIG. 19, administration of Tribody reduced dramatically tumor size compared to the control samples and with significant TGI of 84% on day11 for IM-1222 and 44% on day 18.

Conclusion: Efficacy of Tribody IM-1222 that represent the humanized 5T4_IM53 sequence derived from mAb016 was demonstrated in in-vivo xenograft models with responses ranges from ˜40-84% TGI.

1. An isolated anti-5T4 antibody comprising three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8; or
(ii) the HCDR1, HCDR2, and HCDR3 comprises the amino acid sequences of SEQ ID NOs: 10⁻¹², and the LCDR1, LCDR2, and LCDR3 comprises the amino acid sequences of SEQ ID NOs: 14-16; or
(iii) the HCDR1, HCDR2, and HCDR3 comprises the amino acid sequences of SEQ ID NOs: 18-20, and the LCDR1, LCDR2, and LCDR3 comprises the amino acid sequences of SEQ ID NOs:22-24; or
(iv) the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32; or
(v) the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40; or
(vi) the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48; or
(vii) the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56; or
(viii) the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

2. The anti-5T4 antibody of claim 1, wherein the antibody comprises a heavy chain variable region and a light chain variable region, said heavy chain variable region and light chain variable region comprise the amino acid sequences of SEQ ID NOs: 1 and 5; SEQ ID NOs:9 and 13; SEQ ID NOs: 17 and 21; SEQ ID NOs:25 and 29; SEQ ID NOs:33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs:71-72; SEQ ID NOs:73-74; SEQ ID NOs:75-76; SEQ ID NOs:77-78; SEQ ID NOs:79-80; SEQ ID NOs:81-82; SEQ ID NOs:83-84; SEQ ID NOs:85-86; SEQ ID NOs:87-88; SEQ ID NOs:89-90; SEQ ID NOs: 91-92; SEQ ID NOs:93-94; SEQ ID NOs:95-96; SEQ ID NOs:97-98; SEQ ID NOs:99-100; SEQ ID NOs: 101-102; SEQ ID NOs: 103-104; SEQ ID NOs: 105-106; SEQ ID NOs: 107-108; SEQ ID NOs: 109-110; SEQ ID NOs:111-112; SEQ ID NOs: 113-114; SEQ ID NOs: 115-116; SEQ ID NOs:117-118; SEQ ID NOs: 119-120; SEQ ID NOs: 121-122; SEQ ID NOs: 123-124; SEQ ID NOs: 125-126; SEQ ID NOs: 127-128; SEQ ID NOs: 129-130; SEQ ID NOs: 131-132; SEQ ID NOs: 133-134; SEQ ID NOs: 135-136; SEQ ID NOs: 137-138; SEQ ID NOs: 139-140; SEQ ID NOs: 141-142; SEQ ID NOs:143-144; SEQ ID NOs:145-146; SEQ ID NOs:147-148; SEQ ID NOs: 149-150; or SEQ ID NOs: 151-152.

3. The anti-5T4 antibody of claim 1, wherein the antibody is an IgG, a Fv, a scFv, a Fab, a F(ab′)2, a minibody, a diabody, a triabody, a nanobody, a bispecific antibody, a tri-specific antibody, a multi-specific antibody, or a single domain antibody.

4. The anti-5T4 antibody of claim 3, wherein said IgG is IgG1, IgG2, IgG3, or IgG4.

5. A composition comprising the anti-5T4 antibody of claim 1 and a pharmaceutically acceptable carrier.

6. The composition of claim 5, wherein the anti-5T4 antibody comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8; or
(ii) the HCDR1, HCDR2, and HCDR3 comprises the amino acid sequences of SEQ ID NOs: 10⁻¹², and the LCDR1, LCDR2, and LCDR3 comprises the amino acid sequences of SEQ ID NOs: 14-16; or
(iii) the HCDR1, HCDR2, and HCDR3 comprises the amino acid sequences of SEQ ID NOs: 18-20, and the LCDR1, LCDR2, and LCDR3 comprises the amino acid sequences of SEQ ID NOs:22-24; or
(iv) the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32; or
(v) the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40; or
(vi) the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48; or
(vii) the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56; or
(viii) the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

7. The composition of claim 6, wherein the anti-5T4 antibody comprises a heavy chain variable region and a light chain variable region, said heavy chain variable region and light chain variable region comprise the amino acid sequences of SEQ ID NOs: 1 and 5; SEQ ID NOs:9 and 13; SEQ ID NOs: 17 and 21; SEQ ID NOs:25 and 29; SEQ ID NOs:33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs:71-72; SEQ ID NOs: 73-74; SEQ ID NOs:75-76; SEQ ID NOs:77-78; SEQ ID NOs:79-80; SEQ ID NOs:81-82; SEQ ID NOs:83-84; SEQ ID NOs:85-86; SEQ ID NOs:87-88; SEQ ID NOs:89-90; SEQ ID NOs: 91-92; SEQ ID NOs:93-94; SEQ ID NOs:95-96; SEQ ID NOs:97-98; SEQ ID NOs: 99-100; SEQ ID NOs: 101-102; SEQ ID NOs: 103-104; SEQ ID NOs: 105-106; SEQ ID NOs: 107-108; SEQ ID NOs: 109-110; SEQ ID NOs:111-112; SEQ ID NOs: 113-114; SEQ ID NOs:115-116; SEQ ID NOs:117-118; SEQ ID NOs:119-120; SEQ ID NOs: 121-122; SEQ ID NOs: 123-124; SEQ ID NOs: 125-126; SEQ ID NOs: 127-128; SEQ ID NOs: 129-130; SEQ ID NOs: 131-132; SEQ ID NOs:133-134; SEQ ID NOs: 135-136; SEQ ID NOs: 137-138; SEQ ID NOs: 139-140; SEQ ID NOs:141-142; SEQ ID NOs: 143-144; SEQ ID NOs:145-146; SEQ ID NOs: 147-148; SEQ ID NOs: 149-150; or SEQ ID NOs: 151-152.

8. An isolated polynucleotide sequence encoding the anti-5T4 antibody of claim 1.

9. A vector comprising the polynucleotide sequence of claim 8.

10. A host cell comprising the vector of claim 9.

11. A method of treating a disease in a subject, comprising the step of administering to the subject a composition comprising an effective amount of the anti-5T4 antibody of claim 1.

12. The method of claim 11, wherein the disease is a cancer, an autoimmune disease, GvHD, a viral infection, or a bacterial infection.

13. The method of claim 11, wherein the anti-5T4 antibody comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:2-4, and the LCDR1, LCDR2, and LCDR3 comprises the amino acid sequences of SEQ ID NOs:6-8; or
(ii) the HCDR1, HCDR2, and HCDR3 comprises the amino acid sequences of SEQ ID NOs: 10⁻¹², and the LCDR1, LCDR2, and LCDR3 comprises the amino acid sequences of SEQ ID NOs: 14-16; or
(iii) the HCDR1, HCDR2, and HCDR3 comprises the amino acid sequences of SEQ ID NOs:18-20, and the LCDR1, LCDR2, and LCDR3 comprises the amino acid sequences of SEQ ID NOs:22-24; or
(iv) the HCDR1, HCDR2, and HCDR3 comprises the amino acid sequences of SEQ ID NOs:26-28, and the LCDR1, LCDR2, and LCDR3 comprises the amino acid sequences of SEQ ID NOs:30-32; or
(v) the HCDR1, HCDR2, and HCDR3 comprises the amino acid sequences of SEQ ID NOs:34-36, and the LCDR1, LCDR2, and LCDR3 comprises the amino acid sequences of SEQ ID NOs:38-40; or
(vi) the HCDR1, HCDR2, and HCDR3 comprises the amino acid sequences of SEQ ID NOs:42-44, and the LCDR1, LCDR2, and LCDR3 comprises the amino acid sequences of SEQ ID NOs:46-48; or
(vii) the HCDR1, HCDR2, and HCDR3 comprises the amino acid sequences of SEQ ID NOs:50-52, and the LCDR1, LCDR2, and LCDR3 comprises the amino acid sequences of SEQ ID NOs:54-56; or
(viii) the HCDR1, HCDR2, and HCDR3 comprises the amino acid sequences of SEQ ID NOs:58-60, and the LCDR1, LCDR2, and LCDR3 comprises the amino acid sequences of SEQ ID NOs:62-64.

14. The method of claim 13, wherein the anti-5T4 antibody comprises a heavy chain variable region and a light chain variable region, said heavy chain variable region and light chain variable region comprise the amino acid sequences of SEQ ID NOs: 1 and 5; SEQ ID NOs:9 and 13; SEQ ID NOs: 17 and 21; SEQ ID NOs:25 and 29; SEQ ID NOs:33 and 37; SEQ ID NOs:41 and 45; SEQ ID NOs:49 and 53; or SEQ ID NOs:57 and 61; SEQ ID NOs:65-66; SEQ ID NOs:67-68; SEQ ID NOs:69-70; SEQ ID NOs:71-72; SEQ ID NOs: 73-74; SEQ ID NOs: 75-76; SEQ ID NOs: 77-78; SEQ ID NOs:79-80; SEQ ID NOs: 81-82; SEQ ID NOs: 83-84; SEQ ID NOs:85-86; SEQ ID NOs:87-88; SEQ ID NOs: 89-90; SEQ ID NOs: 91-92; SEQ ID NOs: 93-94; SEQ ID NOs:95-96; SEQ ID NOs: 97-98; SEQ ID NOs: 99-100; SEQ ID NOs: 101-102; SEQ ID NOs: 103-104; SEQ ID NOs: 105-106; SEQ ID NOs: 107-108; SEQ ID NOs: 109-110; SEQ ID NOs: 111-112; SEQ ID NOs: 113-114; SEQ ID NOs: 115-116; SEQ ID NOs: 117-118; SEQ ID NOs:119-120; SEQ ID NOs: 121-122; SEQ ID NOs: 123-124; SEQ ID NOs: 125-126; SEQ ID NOs: 127-128; SEQ ID NOs: 129-130; SEQ ID NOs: 131-132; SEQ ID NOs: 133-134; SEQ ID NOs: 135-136; SEQ ID NOs: 137-138; SEQ ID NOs: 139-140; SEQ ID NOs:141-142; SEQ ID NOs: 143-144; SEQ ID NOs: 145-146; SEQ ID NOs:147-148; SEQ ID NOs: 149-150; or SEQ ID NOs: 151-152.
